(19) United States
(12) Patent Application Publication
(10) Pub. No.: US 2022/0362293 A1
(43) Pub. Date: Nov. 17, 2022

(54) Treatment of Mitochondrial Deficits and Age-Related Diseases Using Blood Products

(71) Applicant: CyteGen Corp., Wauwatosa, WI (US)

(72) Inventors: Ronald Blake Hill, Milwaukee, WI (US); Tomas A. Prolla, Madison, WI (US); Nuray George Ugras, Menlo Park, CA (US)

(73) Assignee: CyteGen Corp., Wauwatosa, WI (US)

(21) Appl. No.: 17/763,005

(22) PCT Filed: Sep. 24, 2020
(86) PCT No.: PCT/US2020/052518

§ 371 Date: Mar. 23, 2022

Related U.S. Application Data
(60) Provisional application No. 62/905,817, filed on Sep. 25, 2019.

(51) Int. Cl. A61K 35/16; A61P 9/02; A61P 3/00

(52) U.S. Cl. CPC A61K 35/16; A61P 9/02; A61P 3/00

(57) ABSTRACT

The present disclosure provides a method of treating mitochondrial and age-related diseases and disorders with exercised blood products. The blood products include circulating factors whose production or secretion into the blood is stimulated by exercise.

### CROSS-REFERENCE TO RELATED APPLICATIONS

This application claims the benefit of priority of U.S. Provisional Application No. 62/905,817, filed Sep. 25, 2019, which is incorporated by reference herein in its entirety for any purpose.

### FIELD

This invention relates to the treatment of mitochondrial dysfunction and age-related diseases and disorders. In particular, the invention relates to the use of blood products, such as blood plasma and blood plasma fractions, that include circulating factors whose production or secretion into the blood is stimulated by exercise.

### BACKGROUND

In eleven years, approximately 71 million Americans (one in five) will be a senior citizen, double the number just fifteen years ago.²³ The Center for Disease Control estimates that approximately 12 million of these Americans will suffer from age-related neurodegenerative diseases with projected costs of over $100 billion annually.²³ Despite the scope and importance of this problem, the molecular basis for most neurodegenerative diseases remains obscure, and there are no FDA approved treatments to effectively treat or prevent age-related disorders.

Aging is caused by a variety of factors,²⁴⁻²⁷ but a common thread is impaired function in the organelle central to cellular metabolism and energy production: the mitochondrion.^{7,28-32} Each cell contains approximately 500-1000 mitochondria (depending on tissue) and each mitochondrion contains its own DNA (approximately 2-10 copies).³³ The mitochondrial DNA accumulate mutations with age in several species including humans³⁴⁻³⁷ and it has long been suggested that mitochondria are central to human aging.^{38,39}

Mitochondria are essential sub-cellular particles involved in a variety of processes, including conversion of nutrients such as carbohydrate and fat into cellular energy in the form of adenosine triphosphate (ATP). Furthermore, mitochondria are involved in cell signaling, cell differentiation and cell death, as well as control of the cell cycle and cell growth. Mitochondrial dysfunction and decay increase with age, may potentially stem from, e.g., oxidative damage to components of mitochondria and mutations to mitochondrial DNA, and may ultimately lead to a variety of diseases.

While mitochondrial dysfunction is tightly linked to aging and cardiac dysfunction, it may be overcome to treat age-related physiological declines and diseases. One example is the reversal of premature aging syndrome in multiple tissues of POLG mutator mice upon forced endurance exercise.²⁰ The POLG mutator mouse is defective in the proofreading capacity of the sole mitochondrial DNA polymerase, POLG, increasing the amount of mtDNA mutations and accelerating the aging process including a severe cardiac defect.¹⁶ Forced endurance exercise appears to reverse the premature aging syndrome, but the mechanism is unknown.

Similarly, while extracellular pathways that signal to stimulate mitochondrial biogenesis are known,^{83,111-113} pathways that stimulate mitophagy are only beginning to be defined.¹¹⁴⁻¹¹⁶ Also, the mechanisms by which mitochondria recognize and segregate damaged DNA (from undamaged) are not known. It likely involves the mitochondrial quality control mechanisms¹¹⁷ being sensitive to mitochondrial DNA damage through loss of membrane potential.¹¹⁸ However, the details of these pathways are also unknown.

### SUMMARY

In accordance with the description, the invention provides methods of treating or preventing a mitochondrial or age-related disease or disorder in a subject in need thereof, comprising administering an effective amount of an exercised blood product to the subject.

In some embodiments, the exercised blood product comprises factors that signal mitochondria to cause regeneration of healthy cells, stem cells and/or progenitor cells.

In some embodiments, the factors comprise small molecules or large molecules.

In some embodiments, the small molecules comprise microRNA (miRNA), metabolites, or steroids.

In some embodiments, the large molecules comprises proteins.

In some embodiments, the proteins have a molecular weight of no more than 30 kDa or no more than 20 kDa.

In some embodiments, the proteins comprise cytokines, hormones or growth factors. In some embodiments, the proteins comprise cytokines. In some embodiments, the cytokines comprise adipokines, chemokines, colony-stimulating factors, interferons, interleukins, monokines, myokines or lymphokines.

In some embodiments, the exercised blood product has been obtained from a blood sample from a donor animal that has been subjected to exercise. In some embodiments, the exercise comprises endurance exercise, rigorous exercise, or resistance exercise.

In some embodiments, the donor animal is young.

In some embodiments, the donor animal is a mammal. In some embodiments, the mammal is a human.

In some embodiments, the method further comprises monitoring the subject for improved mitochondrial fitness or function.

In some embodiments, the subject is a mammal. In some embodiments, the mammal is a human.

In some embodiments, the exercised blood product is administered at least once per week, or at least twice per week, or at least three times per week.

In some embodiments, the exercised blood product is administered for at least 2, 3, 4, 5, 6, 7, 8, 9, or 10 weeks. In some embodiments, the exercised blood product is administered three times per week for at least 4 weeks. In some embodiments, the exercised blood product is administered three times per week for at least 8 weeks.

In some embodiments, the administration is by injection. In some embodiments, the administration is by intravenous injection.

In some embodiments, the exercised blood product comprises plasma components. In some embodiments, the exercised blood product is plasma. In other embodiments, the exercised blood product is a plasma fraction.

In some embodiments, the mitochondrial or age-related disease or disorder is mitochondrial dysfunction.

In some embodiments, the mitochondrial dysfunction comprises a mitochondrial disease, muscle disorder, cardiovascular disease, autoimmune disease, NF-kappaB mediated disease, respiratory disease, neurodegeneration or neuroinflammation, and/or demyelinating neurological disorder.

In some embodiments, the mitochondrial disease is Leber's hereditary optic neuropathy, MELAS (Mitochondrial Encephalomyopathy; Lactic Acidosis; Stroke), MERRF (Myoclonic Epilepsy; Ragged Red Fibers), progressive external opthalmoplegia, Leigh's syndrome, MNGIE (Myopathy and external ophthalmoplegia; Neuropathy; Gastro-Intestinal; Encephalopathy), Kearns-Sayre Syndrome, NARP (Neuropathy, ataxia, and retinitis pigmentosa), Hereditary Spastic Paraparesis, Mitochondrial myopathy, Friedreich ataxia, retinopathia pigmentosa, and/or a form of mitochondrial encephalomyopathy.

In some embodiments, the muscle disorder is sarcopenia, frailty, nemaline myopathy, Spinocerebellar ataxia, Spinal muscular atrophy, or deconditioning from inactivity, hospitalization, and/or any surgical procedure. In some embodiments, the muscle disorder is deconditioning and the treatment reduces the time to recovery.

In some embodiments, the cardiovascular disease is cardiac insufficiency, myocardial infarct, angina pectoris, ischemia and/or reperfusion injury.

In some embodiments, the autoimmune disease is polyarthritis, rheumatoid arthritis, multiple sclerosis, graft-versus-host reactions, juvenile-onset diabetes, Hashimoto's thyroiditis, Grave's disease, systemic Lupus erythematodes, Sjogren's syndrome, pernicious anaemia and chronic active (lupoid) hepatitis, psoriasis, psoriatic arthritis, neurodermatitis, and/or enteritis regionalis Crohn.

In some embodiments, the NF-kappaB mediated disease is progressive systemic sclerodermia, osteochondritis syphilitica (Wegener's disease), cutis marmorata (livedo reticularis), Behcet disease, panarteriitis, colitis ulcerosa, vasculitis, osteoarthritis, gout, artenosclerosis, Reiter's disease, pulmonary granulomatosis, a type of encephalitis, endotoxic shock (septic-toxic shock), sepsis, pneumonia, encephalomyelitis, anorexia nervosa, hepatitis (acute hepatitis, chronic hepatitis, toxic hepatitis, alcohol-induced hepatitis, viral hepatitis, jaundice, liver insufficiency and cytomegaloviral hepatitis), Rennert T-lymphomatosis, mesangial nephritis, post-angioplastic restenosis, reperfusion syndrome, cytomegaloviral retinopathy, adenoviral diseases such as adenoviral colds, adenoviral pharyngoconjunctival fever and adenoviral ophthalmia, AIDS, Guillain-Barré syndrome, post-herpetic or post-zoster neuralgia, inflammatory demyelinising polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumour radiosensitisation, multi-resistance of malignant cells to chemotherapeutic agents (multidrug resistance in chemotherapy), granuloma annulare and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma, and/or Castleman tumour.

In some embodiments, the respiratory disease is asthma, chronic obstructive pulmonary diseases, PDGF induced thymidine uptake of bronchial smooth muscle cells, and/or bronchial smooth muscle cell proliferation.

In some embodiments, the neurodegeneration or neuroinflammation is adrenal leukodystrophy, alcoholism, Alexander's disease, Alper's disease, Alzheimer's disease, amyotrophic lateral sclerosis (Lou Gehrig's Disease), ataxia telangiectasia, Batten disease (also known as Spielmeyer-Vogt-Sjögren-Batten disease), Bovine spongiform encephalopathy, Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjögren-Batten disease (also known as Batten disease), Spinocerebellar ataxia, Spinal muscular atrophy, Steele-Richardson-Olszewski disease, Tabes dorsalis, and/or Toxic encephalopathy.

In some embodiments, the demyelinating neurological disorder is optic neuritis, acute inflammatory demyelinating polyneuropathy, chronic inflammatory demyelinating polyneuropathy, acute transverse myelitis, progressive multifocal leucoencephalopathy, acute disseminated encephalomyelitis, and/or other hereditary disorders (e.g., leukodystrophies, Leber's optic atrophy, and Charcot-Marie-Tooth disease).

In accordance with the description, the invention also provides a pharmaceutical composition for treating, retarding the progression of, delaying the onset of, prophylaxis of, amelioration of, or reducing the symptoms of a mitochondrial dysfunction comprising exercise-derived blood products.

In some embodiments, the mitochondrial dysfunction comprises a mitochondrial disease, muscle disorder, cardiovascular disease, autoimmune disease, NF-kappaB mediated disease, respiratory disease, neurodegeneration or neuroinflammation, and/or demyelinating neurological disorder.

In some embodiments, the mitochondrial disease is Leber's hereditary optic neuropathy, MELAS (Mitochondrial Encephalomyopathy; Lactic Acidosis; Stroke), MERRF (Myoclonic Epilepsy; Ragged Red Fibers), progressive external opthalmoplegia, Leigh's syndrome, MNGIE (Myopathy and external ophthalmoplegia; Neuropathy; Gastro-Intestinal; Encephalopathy), Kearns-Sayre Syndrome, NARP (Neuropathy, ataxia, and retinitis pigmentosa), Hereditary Spastic Paraparesis, Mitochondrial myopathy, Friedreich ataxia, retinopathia pigmentosa, and/or a form of mitochondrial encephalomyopathy.

In some embodiments, the muscle disorder is sarcopenia, frailty, nemaline myopathy, Spinocerebellar ataxia, Spinal muscular atrophy, or deconditioning from inactivity, hospitalization, and/or any surgical procedure. In some embodiments, the muscle disorder is deconditioning and the treatment reduces time to recovery.

In some embodiments, the cardiovascular disease is cardiac insufficiency, myocardial infarct, angina pectoris, ischemia and/or reperfusion injury.

In some embodiments, the autoimmune disease is polyarthritis, rheumatoid arthritis, multiple sclerosis, graft-versus-host reactions, juvenile-onset diabetes, Hashimoto's thyroiditis, Grave's disease, systemic Lupus erythematodes, Sjogren's syndrome, pernicious anaemia and chronic active (lupoid) hepatitis, psoriasis, psoriatic arthritis, neurodermatitis, and/or enteritis regionalis Crohn.

In some embodiments, the NF-kappaB mediated disease is progressive systemic sclerodermia, osteochondritis syphilitica (Wegener's disease), cutis marmorata (livedo reticularis), Behcet disease, panarteriitis, colitis ulcerosa, vasculitis, osteoarthritis, gout, artenosclerosis, Reiter's disease, pulmonary granulomatosis, a type of encephalitis, endotoxic shock (septic-toxic shock), sepsis, pneumonia, encephalomyelitis, anorexia nervosa, hepatitis (acute hepatitis, chronic hepatitis, toxic hepatitis, alcohol-induced hepatitis, viral hepatitis, jaundice, liver insufficiency and cytomegaloviral hepatitis), Rennert T-lymphomatosis, mesangial nephritis, post-angioplastic restenosis, reperfusion syndrome, cytomegaloviral retinopathy, adenoviral diseases such as adenoviral colds, adenoviral pharyngoconjunctival fever and adenoviral ophthalmia, AIDS, Guillain-Barré syndrome, post-herpetic or post-zoster neuralgia, inflammatory demyelinising polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumour radiosensitisation, multi-resistance of malignant cells to chemotherapeutic agents (multidrug resistance in chemotherapy), granuloma annulare and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma, and/or Castleman tumour.

In some embodiments, the respiratory disease is asthma, chronic obstructive pulmonary diseases, PDGF induced thymidine uptake of bronchial smooth muscle cells, and/or bronchial smooth muscle cell proliferation.

In some embodiments, the neurodegeneration or neuroinflammation is adrenal leukodystrophy, alcoholism, Alexander's disease, Alper's disease, Alzheimer's disease, amyotrophic lateral sclerosis (Lou Gehrig's Disease), ataxia telangiectasia, Batten disease (also known as Spielmeyer-Vogt-Sjögren-Batten disease), Bovine spongiform encephalopathy, Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjögren-Batten disease (also known as Batten disease), Spinocerebellar ataxia, Spinal muscular atrophy, Steele-Richardson-Olszewski disease, Tabes dorsalis, and/or Toxic encephalopathy.

In some embodiments, the demyelinating neurological disorder is optic neuritis, acute inflammatory demyelinating polyneuropathy (AIDP), chronic inflammatory demyelinating polyneuropathy (CIDP), acute transverse myelitis, progressive multifocal leucoencephalopathy (PML), acute disseminated encephalomyelitis (ADEM) or other hereditary disorders (e.g., leukodystrophies, Leber's optic atrophy, and Charcot-Marie-Tooth disease).

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1C show that a mutation in the proofreading domain of the mitochondrial DNA polymerase POLG causes premature aging in mice. Images of wildtype (FIG. 1A, +/+) and POLG mutant mice (FIG. 1B, POLG-D257A/D257A substitution) at 13 months old. POLG mice also die prematurely compared to wildtype (FIG. 1C).

FIG. 2 shows that exercised POLG mice (POLG-END) exhibited reversal of the aging syndrome of sedentary animals (POLG-SED). Differences between POLG-END and POLG-SED were seen at 30 weeks, and POLG-END mice were indistinguishable from age-matched wildtype controls at 72 weeks (FIG. 2).

FIGS. 3A-3D show that cardiac function of POLG mice deteriorates with age and is reversed upon exercise or injection with plasma from exercised POLG mice. High-resolution echocardiographic data on young sedentary (3 month), old sedentary (13 month), old exercised (old+exercise, 13 month), and old sedentary IP injected POLG mice (old+IP exer plasma, 13 month) demonstrates improvement in cardiac dysfunction in POLG mice that were exercised or that received plasma from exercised POLG mice. For plasma collection, POLG mice were exercised 3 times week between 2-5 months of age, and plasma was collected immediately after and at 2-hours after an exercise bout. Dialyzed plasma from these two time points was combined and frozen for later injections (100 microliters 3 times/week, for 8 weeks). Images were obtained by a rapid succession of B-mode scans along a single axis over time to further evaluate cardiac function. Heart rate (FIG. 3A), left ventricle mass (FIG. 3B), ejection fraction (FIG. 3C), and fractional shortening (FIG. 3D) were measured over at least three consecutive cardiac cycles and averaged. Left ventricular fractional shortening was calculated as [(LV diameter diastole−LV diameter systole)/LV diameter diastole]×100, and LV mass was calculated by using the formula [1.05×((Posterior Wall diastole+Anterior Wall diastole+LV diameter diastole)³−(LV diameter diastole)³)]. The same person obtained all images and measures. n=5-7 per group each containing males and females, suggesting that sex is not an important variable. All values for old sedentary group were statistically significant in comparison to other groups (ANOVA followed by Tukey test p-value <0.05), except for the comparison of heart rate with IP injected (p<0.11). The red dots in FIGS. 3C and 3D in the old sedentary groups denote outliers (i.e. 1.5 times the interquartile range (IQR)), which only occur because the other data points are so tightly clustered.

FIG. 4 shows the number of gene expression changes in heart, liver, and gastrocnemius muscle of old, sedentary POLG mice is either increased or decreased by exercise and mimicked by injection of exercise-derived plasma (injected intraperitoneally). RNASeq analysis was performed in POLG mouse tissues from old sedentary (13 month), old exercised (old+exercise, 13 months), and old sedentary injected with exercised POLG plasma (“old+IP exercise plasma,” 13 month). Plasma collection was as described for FIG. 3. Of 2811 heart genes increased >1.4 fold by exercise, 2046 (73%) of these genes were also increased >1.4 fold by IP injection of exercise-derived plasma. Of 6282 heart genes decreased >1.4 fold by exercise, 3020 (48%) of these genes were also decreased >1.4 fold by IP injection of exercise-derived plasma. Of 3471 gastrocnemius genes increased >1.4 fold by exercise, 2154 (62%) of these genes were also increased >1.4 fold by IP injection of exercise-derived plasma. Of 6688 gastrocnemius genes decreased >1.4 fold by exercise, 3972 (59%) of these genes were also decreased >1.4 fold by IP injection of exercise-derived plasma. Of 3229 liver genes increased >1.4 fold by exercise, 1222 (38%) of these genes were also increased >1.4 fold by IP injection of exercise-derived plasma. Of 6588 liver genes decreased >1.4 fold by exercise, 2438 (37%) of these genes were also decreased >1.4 fold by IP injection of exercise-derived plasma.

FIG. 5 shows mitochondrial gene expression in the heart upon administration of exercise-derived plasma mimics that of forced endurance exercise. RNASeq analysis and plasma collection was as described for FIGS. 3 and 4. The energy production genes are encoded by both mitochondrial and nuclear genomes and comprise Complexes I-V of the oxidative phosphorylation (OXPHOS) system. In the heart, nuclear-encoded OXPHOS subunit gene expression (CI-CV) decreases significantly with age and is restored to “Young” levels by exercise and IP injection of exercise-derived plasma. In contrast, mitochondrial-encoded OXPHOS genes increase significantly with age in the heart. The expression level remains elevated with exercise, and is restored to “Young” levels by plasma injection. Gene expression values are normalized to “Young” levels and represent the mean±standard error of the mean from n=5-7 animals. Values indicated by an asterisk are statistically significant with p<0.05.

FIG. 6 shows that heart mitochondrial gene expression decreases with age and is reversed upon exercise or injection with plasma from exercised POLG mice. RNASeq analysis was performed in POLG mouse tissues from young sedentary (3 month), old sedentary (13 month), old exercised (old+exercise, 13 months), and old sedentary injected with exercised POLG plasma (“old+IP exercise plasma,” 13 month), presented as bars from left to right for each gene as labeled for Ndufa4. Plasma collection was as described for FIG. 3. Genes shown are nuclear encoded subunits of mitochondrial Complex I and have been associated with Leigh Syndrome, a rare mitochondrial disease. All genes showed a statistically significant difference when comparing young versus old sedentary groups (p<0.05) and when comparing “old sedentary” versus “old+exercise” or “old+IP exercise plasma” (p<0.05).

FIGS. 7A-7B show Seahorse analysis of mitochondrial respiration (FIG. 7A) of WT mouse embryonic fibroblasts (MEFwt) and POLG MEFs (MEFPolg--) showing a defect in maximal respiratory capacity (FIG. 7B). *p-value <0.05. FCCP=carbonyl cyanide p-trifluoromethoxyphenylhydrazone; OCR=oxygen consumption rate. Incubating POLG MEFs in exercised plasma may recover maximal respiration.

### DETAILED DESCRIPTION

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### I. Definitions

As used in the specification and the appended claims, the indefinite articles “a” and “an” and the definite article “the” can include plural referents as well as singular referents unless specifically stated otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

In some embodiments, the term “upon exercise” means during exercise or within a short period of time after the termination of an exercise session or regimen. In other embodiments, the term “upon exercise” means after a certain amount of exercise or after a certain amount of tissue development as a result of exercise. In some embodiments, exercise includes endurance exercise, rigorous exercise, or resistance exercise.

In describing methods of the present invention, the terms “host,” “subject,” “individual,” and “patient” are used interchangeably and refer to any animal in need of such treatment according to the disclosed methods. In some embodiments the animal is a mammal such as, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is a mammal. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The term subject is also meant to include a person or organism of any age, weight or other physical characteristic, where the subjects may be an adult, a child, an infant or a newborn.

By a “young” or “young individual” it is meant an individual that is of chronological age of 40 years old or younger, e.g., 35 years old or younger, including 30 years old or younger, e.g., 25 years old or younger or 22 years old or younger. In some instances, the individual that serves as the source of the young blood product is one that is 10 years old or younger, e.g., 5 years old or younger, including 1-year-old or younger. As such, “young” and “young individual” may refer to a subject that is between the ages of 0 and 40, e.g., 0, 1, 5, 10, 15, 20, 25, 30, 35, or 40 years old. In other instances, “young” and “young individual” may refer to a biological (as opposed to chronological) age such as an individual who has not exhibited a mitochondrial or age-related diseases or disorder.

As used herein, “treatment” refers to any of (i) the prevention of the disease or disorder, or (ii) the reduction or elimination of symptoms of the disease or disorder. Treatment may be affected prophylactically (prior to the onset of disease) or therapeutically (following the onset of the disease). The effect may be prophylactic in terms of completely or partially preventing a disease or symptom thereof and/or may be therapeutic in terms of a partial or complete cure for a disease and/or adverse effect attributable to the disease. Thus, the term “treatment” as used herein covers any treatment of mitochondrial or age-related disease or disorder in an animal, such as a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. The therapeutic agent may be administered before, during or after the onset of disease. The treatment of ongoing disease, where the treatment stabilizes or reduces the undesirable clinical symptoms of the patient, is of particular interest. Such treatment may be performed prior to complete loss of function in the affected tissues. The subject therapy may be administered during the symptomatic stage of the disease, and in some cases after the symptomatic stage of the disease.

The terms “effective amount” or “therapeutically effective amount” refers to an amount of therapeutic agent effective to treat and/or prevent a disease, disorder or unwanted physiological condition in a mammal. In the present invention, an “effective amount” of a blood product may prevent, treat, retard the progression of, delay the onset of, prophlyaxis of, amelioration of, or reduce the disease, disorder, or unwanted condition, such as mitochondrial dysfunction; and/or relieve, to some extent, one or more of the symptoms associated with such a disease, disorder, or condition.

### II. Treating Mitochondrial and Age-Related Diseases and Disorders

Enhancement of mitochondrial fitness or function through, e.g., exercise can have beneficial effects on cells. Moreover, exercise can stimulate the production or secretion of factors (e.g., microRNAs or proteins, such as cytokines) that signal mitochondria to cause regeneration of healthy cells, stem cells and/or progenitor cells. For example, exercise can increase the production of secreted microRNAs or proteins that stimulate mitochondrial biogenesis and selective autophagy of mitochondria (mitophagy, an important mitochondrial quality-control mechanism), which eliminates damaged mitochondria (e.g., those having damaged mtDNA), enriches healthy mitochondria, and supports stem cells and progenitor cells, leading to rejuvenation of the animal.

Dysfunction of adult stem cells and progenitor cells can play an important role in aging. In some embodiments, one or more of the factors promote the health of cells, stem cells and/or progenitor cells, or the maintenance, rejuvenation or regeneration of cells, stem cells and/or progenitor cells.

A. Mitochondrial and Age-Related Diseases and Disorders

Mitochondrial dysfunction or decay can result in damage to cells or tissues of, e.g., the brain, heart, kidney, liver or skeletal muscles, or the cardiovascular, endocrine, nervous or respiratory system. Such damage can cause diseases or disorders associated with aging. For example, mitochondrial dysfunction or decay can lead to neurodegenerative or neuromuscular diseases, such as Alzheimer's disease or Parkinson's disease, and other age-related diseases⁸⁻¹¹, as well as rare mitochondrial diseases that arise from inborn errors in genes encoding mitochondrial proteins that affect 1 in 5,000 individuals^{12,13}. Currently, no effective treatments are available.^{14,15}

Mutations (including single nucleotide polymorphisms and deletions) in the sole mtDNA polymerase, DNA polymerase y, cause a variety of diseases and disorders in humans, including without limitation metabolic diseases (e.g., diabetes), muscle diseases (e.g., mitochondrial myopathy), neuromuscular diseases (e.g., Charcot-Marie-Tooth disease [CMT], Parkinson's disease, ataxia neuropathy syndrome [ANS, including mitochondrial recessive ataxia syndrome {MIRAS} and sensory ataxia neuropathy dysarthria and ophthalmoplegia {SANDO}], and myoclonic epilepsy myopathy sensory ataxia [MEMSA]), neurodegenerative diseases (e.g., Alpers' disease [Alpers-Huttenlocher syndrome {AHS}] and Parkinson's disease), infantile myocerebrohepatopathy spectrum disorders, progressive external ophthalmoplegia (PEO) (including chronic PEO [cPEO], sporadic PEO [sPEO], autosomal dominant PEO [adPEO] and autosomal recessive PEO [arPEO]), tumors, cancers (e.g., testicular cancer), and male infertility. Factors (e.g., proteins, such as cytokines) that phenocopy exercise to overcome a defective mtDNA polymerase can be used to treat mitochondrial and age-related diseases and disorders.

In some embodiments, the mitochondrial and age-related diseases and disorders include diseases and disorders of the brain, eye, heart, liver, kidney, gonad, skeletal muscles, bones, joints, and cardiovascular, digestive, endocrine, respiratory, sensory (e.g., hearing) and central and peripheral nervous systems. In certain embodiments, the mitochondrial and age-related diseases and disorders include cardiovascular diseases (e.g., cardiac dysfunction, heart disease and atherosclerosis), hypertension, metabolic diseases (e.g., diabetes mellitus [e.g., type 2 diabetes] and Leigh's disease), diabetes and deafness, muscle diseases (e.g., mitochondrial myopathy), neuromuscular diseases (e.g., Charcot-Marie-Tooth disease [CMT], Parkinson's disease, ataxia neuropathy syndrome [including MIRAS and SANDO], and myoclonic epilepsy myopathy sensory ataxia [MEMSA]), neurodegenerative diseases (e.g., dementia [e.g., Alzheimer's disease], Alpers' disease, amyotrophic lateral sclerosis [ALS], Huntington's disease and Parkinson's disease), infantile myocerebrohepatopathy spectrum disorders, inflammatory diseases (e.g., arthritis, such as osteoarthritis [which can be caused by, e.g., diabetes]), osteoporosis (bone loss can be induced by, e.g., endocrine disorders, such as diabetes), kyphosis (hunchback), tumors, cancers (e.g., testicular cancer) (tumors and cancers can be caused by, e.g., age-related changes in the endocrine system), cataracts (lens proteins denature and degrade over time, which is accelerated by diseases such as diabetes and hypertension), Leber's hereditary optic neuropathy (LHON), Kearns-Sayre syndrome (KSS), progressive external ophthalmoplegia (PEO) (including cPEO, sPEO, adPEO and arPEO), hearing impairment and loss, anemia, weight loss, decreased subcutaneous fat, male infertility and alopecia (hair loss).

Mitochondrial dysfunction includes, but is not limited to: (1) mitochondrial disease (e.g., Leber's hereditary optic neuropathy, MELAS (Mitochondrial Encephalomyopathy; Lactic Acidosis; Stroke), MERRF (Myoclonic Epilepsy; Ragged Red Fibers), progressive external opthalmoplegia, Leigh's syndrome, MNGIE (Myopathy and external ophthalmoplegia; Neuropathy; Gastro-Intestinal; Encephalopathy), Kearns-Sayre Syndrome, NARP (Neuropathy, ataxia, and retinitis pigmentosa), Hereditary Spastic Paraparesis, Mitochondrial myopathy, Friedreich ataxia, retinopathia pigmentosa, and/or a form of mitochondrial encephalomyopathy); (2) muscle disorders (e.g., sarcopenia, frailty, nemaline myopathy, Spinocerebellar ataxia, Spinal muscular atrophy, or deconditioning from inactivity, hospitalization, and/or any surgical procedure; (3) cardiovascular disease (e.g., cardiac insufficiency, myocardial infarct, angina pectoris, ischemia and/or reperfusion injury); (4) autoimmune diseases (e.g., polyarthritis, rheumatoid arthritis, multiple sclerosis, graft-versus-host reactions, juvenile-onset diabetes, Hashimoto's thyroiditis, Grave's disease, systemic Lupus erythematodes, Sjogren's syndrome, pernicious anaemia and chronic active (lupoid) hepatitis, psoriasis, psoriatic arthritis, neurodermatitis, and/or enteritis regionalis Crohn); (5) NF-kappaB mediated diseases (e.g., progressive systemic sclerodermia, osteochondritis syphilitica (Wegener's disease), cutis marmorata (livedo reticularis), Behcet disease, panarteriitis, colitis ulcerosa, vasculitis, osteoarthritis, gout, artenosclerosis, Reiter's disease, pulmonary granulomatosis, a type of encephalitis, endotoxic shock (septic-toxic shock), sepsis, pneumonia, encephalomyelitis, anorexia nervosa, hepatitis (acute hepatitis, chronic hepatitis, toxic hepatitis, alcohol-induced hepatitis, viral hepatitis, jaundice, liver insufficiency and cytomegaloviral hepatitis), Rennert T-lymphomatosis, mesangial nephritis, post-angioplastic restenosis, reperfusion syndrome, cytomegaloviral retinopathy, adenoviral diseases such as adenoviral colds, adenoviral pharyngoconjunctival fever and adenoviral ophthalmia, AIDS, Guillain-Barré syndrome, post-herpetic or post-zoster neuralgia, inflammatory demyelinising polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumour radiosensitisation, multi-resistance of malignant cells to chemotherapeutic agents (multidrug resistance in chemotherapy), granuloma annulare and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma, and/or Castleman tumour); (6) respiratory diseases (e.g., asthma, chronic obstructive pulmonary diseases, PDGF induced thymidine uptake of bronchial smooth muscle cells, and/or bronchial smooth muscle cell proliferation); (7) neurodegeneration or neuroinflammation (e.g., adrenal leukodystrophy, alcoholism, Alexander's disease, Alper's disease, Alzheimer's disease, amyotrophic lateral sclerosis (Lou Gehrig's Disease), ataxia telangiectasia, Batten disease (also known as Spielmeyer-Vogt-Sjögren-Batten disease), Bovine spongiform encephalopathy, Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjögren-Batten disease (also known as Batten disease), Spinocerebellar ataxia, Spinal muscular atrophy, Steele-Richardson-Olszewski disease, Tabes dorsalis, and/or Toxic encephalopathy); and/or (8) demyelinating neurological disorders (e.g., optic neuritis, acute inflammatory demyelinating polyneuropathy (AIDP), chronic inflammatory demyelinating polyneuropathy (CIDP), acute transverse myelitis, progressive multifocal leucoencephalopathy (PML), acute disseminated encephalomyelitis (ADEM) or other hereditary disorders (e.g., leukodystrophies, Leber's optic atrophy, and Charcot-Marie-Tooth disease)).

Reactive byproducts of aerobic respiration in mitochondria, such as free radicals, potentially may over time cause damage (e.g., oxidative damage) to lipids, proteins, RNA and DNA in mitochondria and elsewhere in the cell, resulting in mitochondrial dysfunction and decay, apoptosis and age-related decline.

B. Factors that Improve Mitochondrial Fitness and Function

Retardation of mitochondrial dysfunction or decay through, e.g., exercise, may retard age-related decline. In certain embodiments, one or more of the factors whose production or secretion is induced by exercise (and/or exposure to stress, such as repetitive or continual mild stress) retard, curtail, reverse or prevent mitochondrial dysfunction, impairment, decay or disorders, and/or age-related decline, functional deficits or disorders.

The factors that can have beneficial effects on mitochondria and/or cells, and/or can have anti-aging effects, can be proteins or non-protein biomolecules. In some embodiments, the factors include small molecules (e.g., microRNA, metabolites or steroids) or large molecules (e.g., polypeptides or proteins). In some embodiments, the factors include proteins or non-protein biomolecules that have a molecular weight of no more than about 30, 25, 20, 15 or 10 kDa (e.g., no more than about 20 kDa). In certain embodiments, the protein factors include cytokines, including without limitation adipokines, chemokines, colony-stimulating factors, interferons, interleukins, monokines, myokines and lymphokines. Cytokines play an important role in intercellular communication and can act in an endocrine manner. In some embodiments, the cytokine factors include fractalkine [aka chemokine (C-X3-C motif) ligand 1 (CX3CL1)], growth differentiation factor 11 (GDF11), interleukin 10 (IL-10) and IL-15. In further embodiments, the factors include hormones, such as irisin and meteorin-like (Metml) protein. In yet further embodiments, the factors include growth factors. There may be some overlap in the terminology of cytokines, hormones and growth factors. For instance, growth differentiation factors (aka bone morphogenetic proteins) may be regarded as cytokines or growth factors.

Some embodiments of the disclosure relate to a method of treating or preventing mitochondrial or age-related diseases or disorders in a subject in need thereof, comprising administering to the subject an effective amount of a blood product, wherein the blood product comprises factors that signal mitochondria to cause regeneration of healthy cells, stem cells and/or progenitor cells. In some embodiments, the blood product has been obtained from a blood sample from an animal that has been subjected to exercise. In some embodiments, the animal is a mammal, such as a human.

C. Blood Products

“Blood products” as used herein include, but are not limited to, blood, plasma, serum, and other products derived from a blood sample. Blood products obtained from blood samples from an animal (e.g., human or other mammal) subjected to exercise (and/or exposed to stress, such as repetitive or continual mild stress) may be referred to as “exercised blood products” and contain the beneficial factors that can be used directly or otherwise developed into therapeutics for the treatment of mitochondrion-associated diseases and disorders and aging-associated diseases and disorders.

In practicing the subject methods, a blood product, such as an exercised blood product, is administered to the individual in need thereof. Embodiments of the invention include administering a blood product comprising plasma components. By a “blood product comprising plasma components,” it is meant any product derived from blood that comprises plasma (e.g. whole blood, blood plasma, or fractions thereof). The term “plasma” is used in its conventional sense to refer to the straw-colored/pale-yellow liquid component of blood composed of about 92% water, 7% proteins such as albumin, gamma globulin, anti-hemophilic factor, and other clotting factors, and 1% mineral salts, sugars, fats, hormones and vitamins. Non-limiting examples of plasma-comprising blood products suitable for use in the subject methods include whole blood treated with anti-coagulant (e.g., EDTA, citrate, oxalate, heparin, etc.), blood products produced by filtering whole blood to remove white blood cells (“leukoreduction”), blood products consisting of plasmapheretically-derived or apheretically-derived plasma, fresh-frozen plasma, blood products consisting essentially of purified plasma, and blood products consisting essentially of plasma fractions. In some instances, plasma product that is employed is a non-whole blood plasma product, by which is meant that the product is not whole blood, such that it lacks one or more components found in whole blood, such as erythrocytes, leukocytes, etc., at least to the extent that these components are present in whole blood. In some instances, the plasma product is substantially, if not completely, acellular, where in such instances the cellular content may be 5% by volume or less, such as 1% or less, including 0.5% or less, where in some instances acellular plasma fractions are those compositions that completely lack cells, i.e., they include no cells.

Methods of collection of plasma comprising blood products from donor animals, such as human donors, are well-known in the art. See, e.g., AABB TECHNICAL MANUAL, (Mark A. Fung, et al., eds., 18th ed. 2014). In one embodiment, donations are obtained by venipuncture. In another embodiment, the venipuncture is only a single venipuncture. In another embodiment, no saline volume replacement is employed. In a preferred embodiment, the process of plasmapheresis is used to obtain the plasma comprising blood products. Plasmapheresis can comprise the removal of a weight-adjusted volume of plasma with the return of cellular components to the donor. In the preferred embodiment, sodium citrate is used during plasmapheresis in order to prevent cell clotting. The volume of plasma collected from a donor is preferably between 690 to 880 mL after citrate administration, and preferably coordinates with the donor's weight.

The blood product can be administered to the subject in need thereof via any suitable mode (e.g., parenterally, such as intramuscularly, subcutaneously, intravenously or intraperitoneally). In some embodiments, the blood product is administered by injection, such as intraperitoneal injection.

Aspects of the methods of the inventions described herein include treatment of a subject with a plasma comprising blood product, such as a blood plasma or plasma fraction. An embodiment includes treatment of a human subject with a plasma comprising blood product. One of skill in the art would recognize that methods of treatment of subjects with plasma comprising blood products are recognized in the art. By way of example, and not limitation, one embodiment of the methods of the inventions described herein is comprised of administering fresh frozen plasma to a subject. In one embodiment, the plasma comprising blood product is administered immediately, e.g., within about 12-48 hours of collection from a donor, to the subject. In such instances, the product may be stored under refrigeration, e.g., 0-10° C. In another embodiment, fresh frozen plasma is one that has been stored frozen (cryopreserved) at −18° C. or colder. Prior to administration, the fresh frozen plasma is thawed and once thawed, administered to a subject 60-75 minutes after the thawing process has begun. Each subject preferably receives a single unit of fresh frozen plasma (200-250 mL), the fresh frozen plasma preferably derived from donors of a pre-determined age range. In one embodiment of the invention, the fresh frozen plasma is donated by (derived from) young individuals. In another embodiment of the invention, the fresh frozen plasma is donated by (derived from) donors of the same gender. In another embodiment of the invention, the fresh frozen plasma is donated by (derived from) donors of the age range between 18-22 years old.

In an embodiment of the invention, the plasma comprising blood products are screened after donation by blood type. In another embodiment of the invention, the plasma comprising blood products are screened for infectious disease agents such as HIV I & II, HBV, HCV, HTLV I & II, anti-HBc per the requirements of 21 CFR 640.33 and recommendations contained in FDA guidance documents.

The blood product is administered in a suitable dose and frequency (e.g., at least once daily). In some embodiments, the blood product is administered 2, 3, 4, 5, 6, or 7 times a week. In some embodiments, the blood product is administered at least twice a week or at least three times a week. In some embodiments, the blood product is administered for at least 1, 2, 3, 4, 5, 6, 7, 8, 9, or 10 weeks. In some embodiments, the blood product is administered three times per week for 4 weeks. In other embodiments, the blood product is administered three times per week for 8 weeks.

D. The POLG Mouse

The POLG mouse has a profound mitochondrial syndrome with reduced life span and premature aging¹⁶. The premature aging of the POLG mouse (aka mitochondrial mutator mouse) derives from fidelity of mitochondrial DNA replication being impaired through a homozygous knock-in mutation that removes the exonuclease proofreading activity of POLG, the sole mtDNA polymerase.

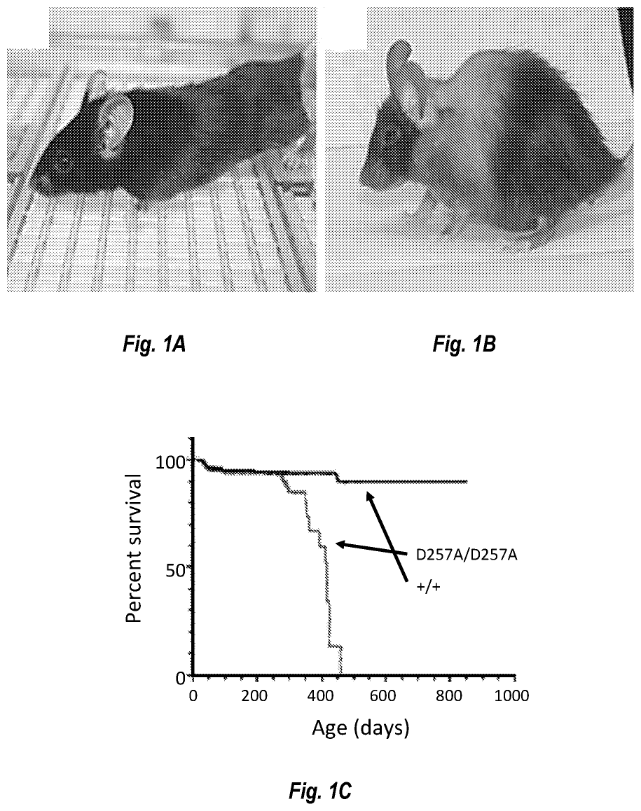

This lesion resulted in a residue substitution (D257A) in the highly conserved exonuclease domain of the sole mammalian mtDNA polymerase, POLG, impairing its proofreading ability^{40,41} Homozygous knock-in mice with the POLG-D257A substitution (POLG mice) showed a profound aging syndrome with reduced life span (FIGS. 1A-C) and premature aging including weight loss, decreased subcutaneous fat, alopecia, kyphosis, osteoporosis, anemia, reduced fertility, and cardiac dysfunction.¹⁶ Similar findings were independently reported by others confirming the central role of POLG to this aging syndrome.⁴² Recent studies have shown that this syndrome is due in part to adult stem cell dysfunction,⁴³ which is thought to play a central role in the normal aging process.²⁷

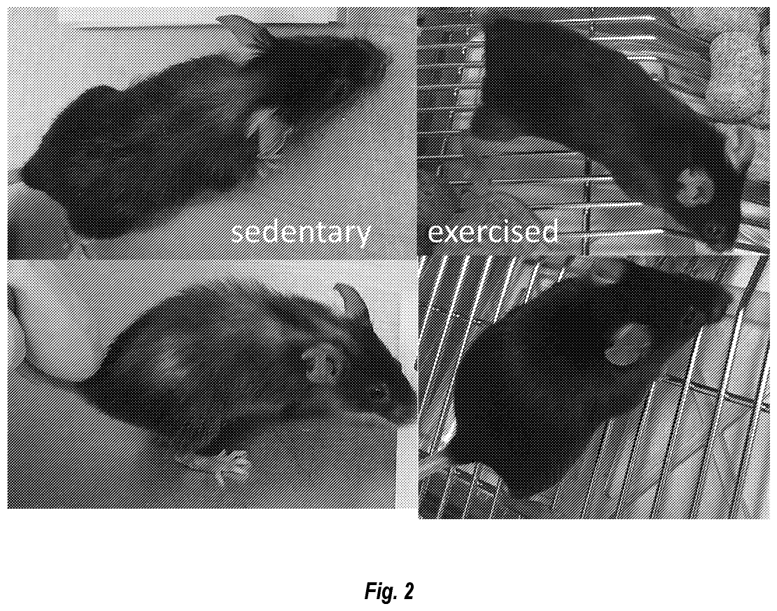

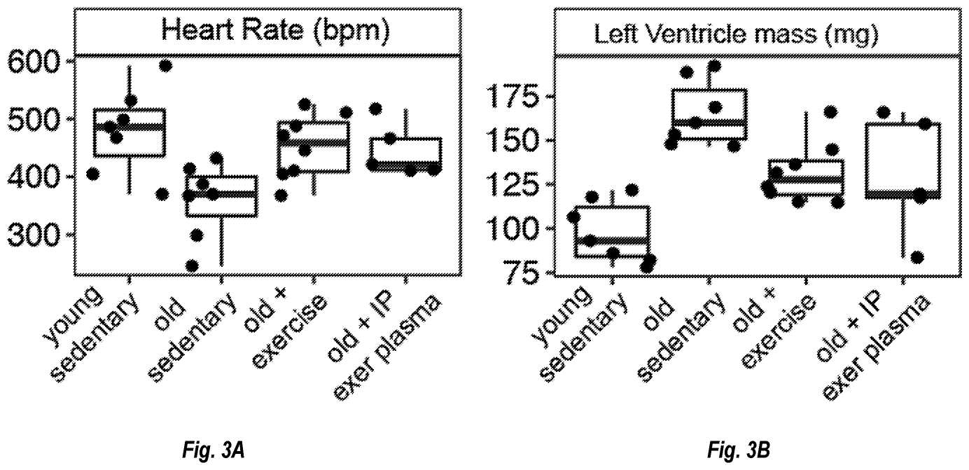

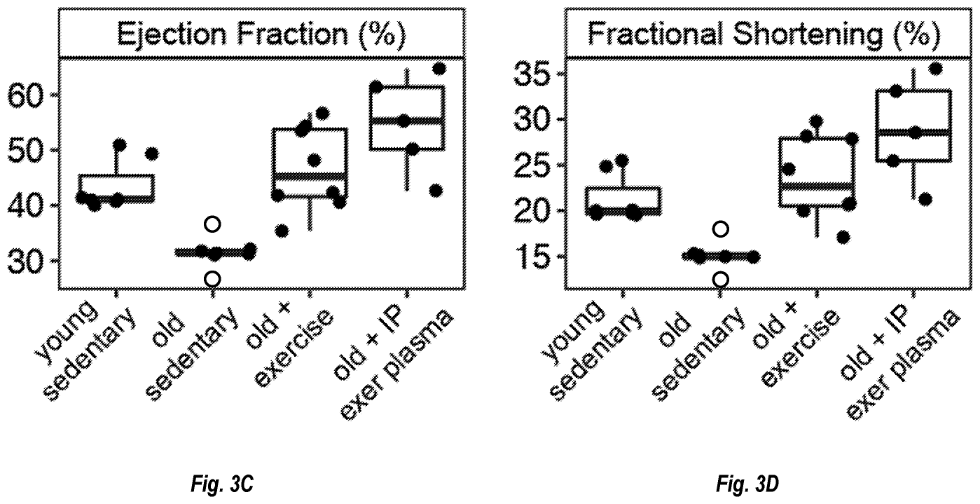

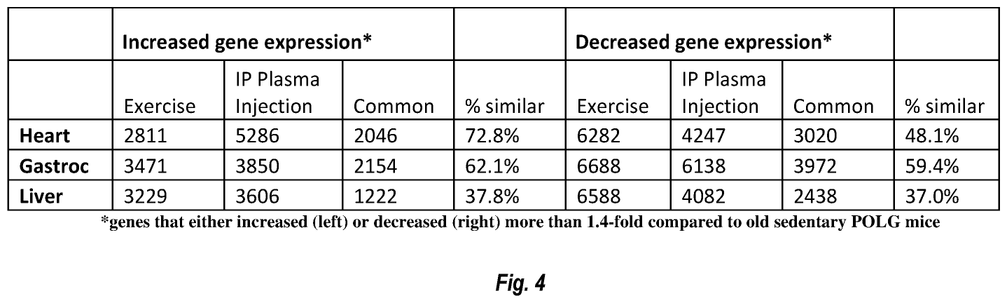

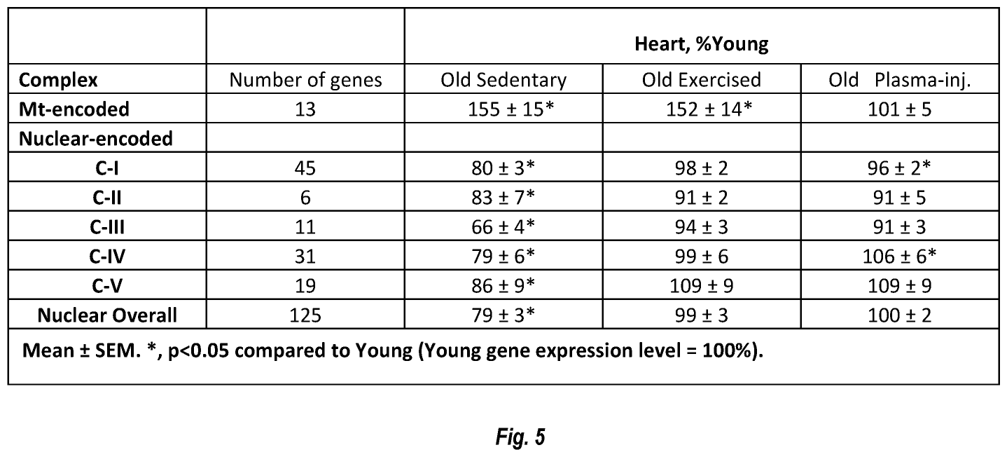

Recently, endurance exercise was shown to reverse this aging syndrome in POLG mice including a return to normal lifespan.²⁰ Phenotypically and histologically exercised mice are indistinguishable from wild type mice, even though they still harbor the genetic lesion. Remarkably, only five months of endurance exercise of POLG mice (run for 45 min at 15 m/min, 3×/week), improved mitochondrial function (FIG. 2) and reduced pathologies in all tissues including the brain. Importantly, the inventors repeated the exercise experiments and saw an exercise benefit to cardiac function, since old exercised POLG mice show cardiac parameters more similar to young POLG mice (FIGS. 3A-3D, discussed below). It is thought that exercise induces the secretion of circulating factors that act to maintain mitochondrial fitness through the selective removal of damaged mitochondria. Supporting the existence of circulating factors, old sedentary POLG mice display improved cardiac function following a series of injections of plasma collected between 0-2 hours after exercise (FIGS. 3A-3D, discussed below). Gene expression profiling (RNAseq) analysis shows that both aerobic exercise and injections of plasma from exercised mice alter the expression of many genes including mitochondrial genes, in heart, liver, and gastrocnemius (FIGS. 4-6, discussed below).

POLG is highly conserved between mice and human and over 200 mutations in this polymerase have been associated with human disease,¹⁷⁻¹⁹ supporting the translation of mouse discoveries into humans. Endurance exercise of POLG mutant mice reversed the early-onset aging syndrome.²⁰⁻²² Analyses of multiple tissues in the exercised mice suggested that endogenous circulating factors may be responsible.²⁰

### EXAMPLES

### Example 1. Obtaining Exercised/Sedentary Plasma

To provide blood for testing, ninety-six POLG animals (B6 strain) are maintained in an exercise regimen from 3 to 6 months of age with forced treadmill running at 15 m/min for 45 minutes 3 times/week (Eco 3/6 treadmill; Columbus Instruments).¹⁴ These animals, and a group (n=48) of sedentary animals, are sacrificed at 6 months of age and blood collected. Many exercise-induced changes in protein levels peak in blood during exercise or shortly thereafter in both humans and mice.⁶²⁻²⁸ Blood is collected and pooled at 0 and 2 hours post-exercise. The blood is separated into plasma and immediately frozen for later use in reinjection experiments and cell-based assays.

A standard operating procedure is used for blood and tissue harvesting that controls critical process variables such as time of sacrifice in relation to feeding, process/storage containers, order of tissue harvest time, centrifugation speed, temperature of storage and dialysis to remove small molecular weight (non-protein) compounds. On average, 500 μL of plasma can be obtained from one mouse. Plasma from individual mice collected at the two time points may be later pooled prior to reinjection or cell based experiments. This protocol yields approximately 48 mL/24 mL of exercised/sedentary plasma.

### Example 2. Effect of Plasma from Exercised Mice on Cardiac Function

To test the hypothesis that exercise induces the secretion of circulating factors that act systemically to reverse aging, plasma from exercised and sedentary POLG mice was collected and injected into sedentary POLG mice. The injection protocol (150 ml, 3×/week for 4 weeks) was designed to mimic the endurance exercise protocol that causes aging prevention. After the completion of the injection protocol, sedentary and exercised POLG mice were tested for altered cardiovascular function. Tissues were also harvested from heart, liver, cortex, and gastrocnemius with RNA isolated for RNASeq transcriptome analyses targeted on mitochondrial pathways to highlight compensatory responses.

FIGS. 3A-3D demonstrate that cardiac function of POLG mice deteriorates with age and is reversed upon exercise or injection with plasma from exercised POLG mice. High-resolution echocardiographic data on young sedentary (3 month), old sedentary (13 month), and old exercised (old+exercise, 13 month), and old sedentary IP injected POLG mice (old+IP exer plasma, 13 month) demonstrates improvement in cardiac dysfunction in POLG mice that were exercised or received plasma from exercised POLG mice.

For plasma collection, POLG mice were exercised 3× week between 2-5 months of age, and plasma was collected immediately after and 2-hours after an exercise bout. Dialyzed plasma from these two time points was combined and frozen for later injections (100 microliters 3 times/week, for 8 weeks). Images were obtained by a rapid succession of B-mode scans along a single axis over time to further evaluate cardiac function. Heart rate (FIG. 3A), left ventricle mass (FIG. 3B), ejection fraction (FIG. 3C), and fractional shortening (FIG. 3D) were measured over at least three consecutive cardiac cycles and averaged. Left ventricular fractional shortening was calculated as [(LV diameter diastole−LV diameter systole)/LV diameter diastole]×100 and LV mass was calculated by using the formula [1.05×((Posterior Wall diastole+Anterior Wall diastole+LV diameter diastole)³−(LV diameter diastole)³)]. The same person obtained all images and measures. n=5-7 per group each containing males and females suggesting that sex is not an important variable. All values compared to old sedentary were statistically significant (ANOVA followed by Tukey test p-value <0.05) except for heart rate with IP injected (p<0.11).

Exercised plasma was found to improve cardiac function in several, but not all, echocardiographic measures (left ventricular mass, wall thickness, Isovolumic relaxation time, others). See FIGS. 3A-3D. Blood hemoglobin and mean corpuscular volume were also improved upon exercise, but not white blood cell count. In a parallel study, exercised plasma was heat-treated to test whether the exercise benefit derived from proteins or not.

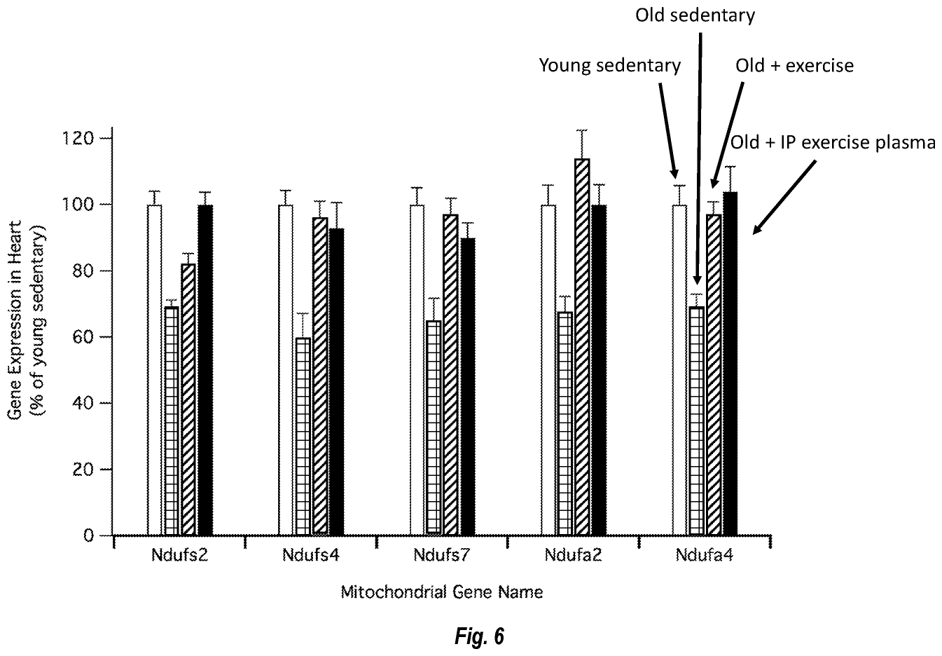

By RNA Sequencing (RNA-Seq), several pathways were identified to be up- or down-regulated upon age and recovered to some extent by exercise or plasma injections. See FIGS. 4-6. Overall gene expression changes show difference upon age that are reversed in many instances upon exercise of plasma injections. Heart mitochondrial gene expression decreases with age and is reversed upon exercise or injection with plasma from exercised POLG mice (FIG. 6). RNASeq analysis was performed in POLG mouse tissues from young sedentary (3 month), old sedentary (13 month), old exercised, and old sedentary injected with exercised POLG plasma (13 month). Plasma collection was as described for FIGS. 3A-3D. Genes shown are nuclear encoded subunits of mitochondrial Complex I, and have been associated with Leigh Syndrome, a rare mitochondrial disease. All genes shown statistically significant on young vs old sedentary comparison (p<0.05), and old sedentary vs old exercised and old exercised plasma injected (p<0.05). The results support that exercise induces the secretion of circulating factors that can mitigate the POLG mutation and presumably act by improving mitochondrial fitness.

### Example 3. The Effect on Plasma from Exercised POLG Mice on Cardiac and Metabolic Dysfunction of Sedentary POLG Mice

To determine if the plasma from exercised mice can improve critical health parameters and mitochondrial function in sedentary POLG mice, 40 sedentary 10-month old POLG animals are divided into groups of ten animals. Groups contain equal numbers of males and females, allowing for the determination of sex-specific effects.

Each mouse receives three IV injections of 100 μl of plasma/week to mimic endurance exercise 3×/week, for eight weeks. The frozen plasma collected from mice in Example 1 is used. Based on literature precedent⁶⁹ and previous parabiosis related experiments,⁴⁹ the most efficacious route appears to be IV. One group receives injections from plasma of exercised mice, while the second group receives plasma injections from the sedentary mice. A third group receives heat-inactivated plasma injections from exercised mice. A fourth group receives saline injections as the control group.

Animals are tested for multiple parameters following treatment: Metabolic capacity, spontaneous activity and cardiac function at 12 months of age. Metabolic capacity us tested using a Comprehensive Lab Animal Monitoring System (CLAMS, Columbus instruments), which allows for the simultaneous measurement of O₂, CO₂, food consumption respiratory exchange ratio (RER), and activity tracking. Animals are acclimated to the metabolic chamber for approximately 24 hours prior to the initiation of data collection, which will occur over a 48 hour period.

12-month old POLG mice display a major reduction in metabolic capacity and spontaneous physical activity as determined by the CLAMS system compared to wild-type animals at 10-12 months of age.⁵ These effects are clearly age-related, as no differences are observed in a comparison of 2-3 month POLG mice with age-matched wild-type littermates.

Cardiac testing is performed at the UW Cardiovascular Physiology Core Facility, using high-resolution echocardiography as in FIG. 3. Multiple parameters are measured, including ejection fraction, fractional shortening, cardiac chamber dimensions, Myocardial Performance Index (MPI). Following cardiac testing, animals are sacrificed with blood and tissues collected, weighed, and frozen for future analyses.

To assess mitochondrial function, OROBOROS high resolution respirometry is used on permeabilized fresh heart tissue and skeletal muscle tissue (gastrocnemius). This system for mitochondrial functional measurement provides high sensitivity, low instrument background, and precise temperatures, allowing titration of substrates, inhibitors and uncouplers without the need to isolate mitochondria.^{71,72} In humans, impaired mitochondrial respiration as determined by high resolution respirometry parallels decreases in muscle performance and aerobic fitness.⁷³ This system was recently used to detect a major decrease in mitochondrial respiratory capacity in aged POLG mice (heart and brain). Mitochondrial oxygen consumption under various conditions is determined using different substrates/inhibitors and is normalized to mitochondrial mass markers, such as citrate synthase.⁷³

Since exercised plasma reverses cardiac dysfunction in POLG mice, it is anticipated that plasma from exercised POLG mice will fully or partially reverse the reduced metabolic and spontaneous activity level, as well as mitochondrial dysfunction in POLG mice. It is also anticipated that heat inactivated plasma will have no effect.

In addition, whether this aging syndrome is reversed at the molecular level in skeletal muscle and brain evaluating mutational load of mtDNA, expression of mitochondrial respiratory chain component proteins,⁷⁴ proteins involved in mitochondrial fusion and fission,⁷⁵ as well as pro-apoptotic proteins are evaluated as described in Example 4.

### Example 4. The Effect on Plasma from Exercised POLG Mice on Mitochondrial Function in POLG Cells

To determine whether the exercised plasma improves mitochondrial function of POLG cells, immortalized (MEFs) from POLG mice and control (WT) mice are used.

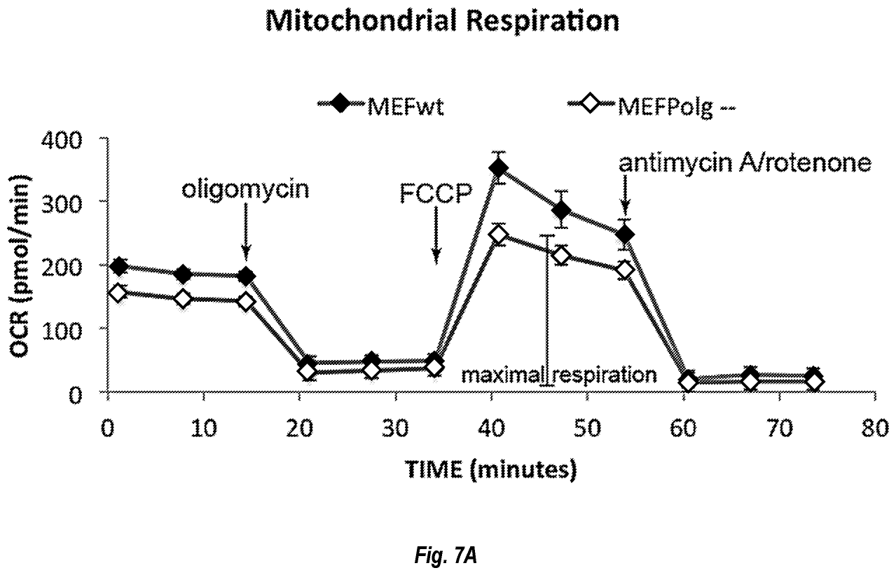

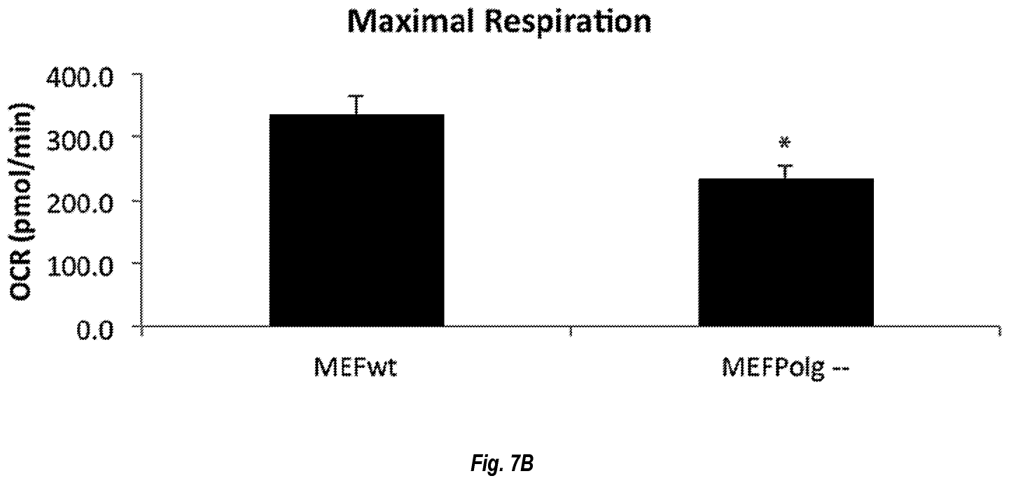

The primary readout for improved mitochondrial function is determination of mitochondrial respiration. Data from Example 2 (FIG. 7) and the work of others¹⁻⁴ show that MEFs have sufficient oxidative phosphorylation for these purposes. Improvement in mitochondrial respiration can arise from increases in mitochondrial mass and/or quality, which will be assessed in secondary assays that measure mtDNA copy number and deletion frequency using a high resolution droplet digital PCR assay described below.

To distinguish between mitochondrial biogenesis and mitophagy, protein expression are measured by several well-established markers⁸³⁻⁸⁶. Mitophagy is more directly measured by transfecting these cells with the mtKeima, a pH-sensitive fluorescent protein that can robustly detect mitophagy.⁸⁷⁻⁸⁹

To evaluate whether plasma from exercised, not sedentary, mice improves mitochondrial function, mitochondrial respiration is measured using the maximal respiratory capacity with the Seahorse XF96 instrument.^{90,91} Measurements are made on cells cultured in 2-10% exercised, exercised/heat-treated, and sedentary plasma and normalized to total cellular protein by a Bradford assay.⁹² Maximal respiratory capacity is determined upon addition of the uncoupler carbonyl cyanide p-trifluoromethoxyphenylhydrazone (FCCP) followed by shutting down the electron transport chain by simultaneous addition of rotenone (Complex I inhibitor) and antimycin A (Complex III inhibitor) and is an excellent measure of peak mitochondrial performance. Proton leak is measured as well in the Seahorse experiment and can be subtracted from maximal respiration if >10%. MEFs do utilize mitochondrial respiration as shown in FIG. 7, which is attenuated in POLG MEFs indicating a suitable dynamic range for evaluating plasma. Cells may also be cultured in galactose or other substrates (2-deoxyglucose, lipids, etc.), which force cells to rely almost exclusively on ATP from mitochondrial respiration.^{93,94} This approach has also been successful in evaluating mitochondrial function in human disease, drug discovery and toxicity.⁹⁵⁻⁹⁷

A dose-response curve of maximal respiratory capacity as a function of plasma concentration is analyzed in relation to controls for plate and assay variability including random errors associated with the day of the experiment and the plates as described.⁹⁸ A Z′-score is calculated based on the maximal response from controls and exercised plasma. Controls are known agonists (GW1516, bezafibrate, rosiglitazone—PPAR, AICAR-AMPK, quinones-PGC-1α) and antagonists (GSK0660, GW9662—PPARs) of mitochondrial biogenesis and mitophagy⁹⁹ and are commercially available. Each respiration experiment requires 200 μL of medium/well. For ten technical replicates and at least three biological replicates requires 1.8 mL of plasma for each condition.

Improvements in mitochondrial respiration derive from either increases in mitochondrial mass and/or quality. To evaluate this, mtDNA copy number and deletions are measured. POLG mice and cells have increased mtDNA mutations and deletions^{100,101} that returns to normal levels upon endurance exercise, presumably through improved mitochondrial quality control.²⁰ mtDNA copy number and deletions may be measured by a droplet digital PCR based method, which allows detection of rare mtDNA deletions in mtDNA102 that occur with frequencies of 1×10-8. The droplet digital PCR method is quantitative, eliminates multiple rounds of PCR with nested primers, and is independent of PCR efficiency.¹⁰³ It also eliminates the need for reference samples and allows a more accurate determination of mtDNA copy number. The superiority of the droplet digital approach has recently been confirmed by others.^{104,105}

The method involves enriching, amplifying, and analyzing the mtDNA. The enrichment step uses the restriction endonuclease TaqI to selectively digest wild-type (WT) molecules at 29 sites spread across a large portion of the mtDNA major arc. After digestion, only mutant mtDNA survives the workflow and then is distributed into approximately 20,00 water-in-oil emulsion droplets (about 1 nL) for normal PCR amplification. The concentration of molecules within the droplets is adjusted to give a single genome in each droplet, which each deletion to be amplified without bias and eliminates template switching and preferential amplification of short templates that are common to bulk PCR. Following amplification, high-resolution quantification of deletions is accomplished using TaqMan reporter chemistry and droplet digital PCR. Poisson statistics are applicable given the droplet uniformity and the average number of deletion-bearing molecules per droplet and the absolute concentration of mutant molecules is calculated with high precision and accuracy.¹⁰³

POLG MEF cells incubated with exercised, sedentary, and exercised/heat-treated plasma are analyzed for mtDNA copy number and deletions using this method. Removal of damaged mitochondria (without compensatory biogenesis) is expected to show reduced levels of mtDNA. However, increases in mtDNA copy number do not address whether plasma treatment might stimulate removal of damaged mitochondria to retain a healthy pool. For this reason, plasma treatments that show an equal or increased mtDNA copy number will be assessed for mitochondrial protein expression using a semi-automated, quantitative Western blot analysis of 16 proteins involved in mitochondrial fitness (see Table 1).

TABLE 1
Protein markers for mitochondrial fitness

| Antibody | Function |
|---|---|
| GAPDH | Housekeeping |
| FIS1 | Mitochondrial dynamics |
| DRP1 | |
| MFF | |
| MFN1 | |
| MFN2 | |
| OPA1 | |
| AMMPK ALPHA | Mitochondrial biogenesis |
| POLG | |
| TOM20 | |
| LC3B | Mitophagy |
| GABARAP | |
| P62/SQSTM1 | |
| NDUFB8 | Electron Transport Chain |
| CYTC | |
| UQCRC2 | |

For these experiments, 50,000 cells are seeded in 6-well plates (2 mL medium) and cultured for 24-48 hours in 2-10% plasma levels. Collected cells from each well are split into half, which is sufficient for mtDNA and protein analyses. For six technical and three biological replicates, 9 mL of exercised and 4.5 mL of sedentary plasma is used. Protein levels from this assay should inform on mitochondrial mass by measuring amounts of electron transport chain proteins, which is standard in the field.¹⁰⁶

Decreases in mitochondrial protein levels by Western blot may indicate that mitochondrial biogenesis is decreased or mitophagy is increased. Further, the processes may be adjusted as appropriate to detect changes to mitochondrial protein levels.

Mitochondrial outer membrane proteins are degraded by both mitophagy and the proteasome. For these reasons, mitophagy is also measured directly by transfecting cells with mtKeima, a pH-sensitive fluorescent protein targeted to the mitochondrial matrix.⁸⁷⁻⁸⁹ Using a mitochondrial targeting sequence from COX VIII, mtKeima is localized in the mitochondrial matrix where the pH is slightly basic (˜pH 8) and the protein fluorescence is green (Katayama et al., 2011). The mitochondrial matrix pH contrasts with the acidic environment of the lysosome (˜pH 4.5), where the protein fluorescence is red. The pH difference between the mitochondrial and lysosome, and Keima's resistance to lysosomal degradation, provides a direct measure of mitophagy by confocal microscopy.^{87,88,107-109}

In the alternative, cells are incubated longer for 48 to 96 hours (e.g., 48, 72, or 96 hours) with appropriate changes of the supplemented medium. For example, a defined medium may also be used, such as “plasmax,” which is reported to provide a more realistic metabolic fidelity in cell culture.¹¹⁰

In another alternative, experiments with tissue homogenates from exercised versus sedentary POLG mice are conducted. The tissue homogenates will be prioritized based on which tissues show improved recovery of mtDNA upon exercise. Using tissue homogenates has been very successful in the past at identifying low abundance circulating factors.⁷⁷⁻⁸²

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

### LITERATURE CITED

1. Nemoto, S., Combs, C. A., French, S., Ahn, B.-H., Fergusson, M. M., Balaban, R. S. & Finkel, T. The mammalian longevity-associated gene product p66shc regulates mitochondrial metabolism. J. Biol. Chem. 281, 10555-10560 (2006).
2. Soliman, M. A., Abdel Rahman, A. M., Lamming, D. W., Birsoy, K., Pawling, J., Frigolet, M. E., Lu, H., Fantus, I. G., Pasculescu, A., Zheng, Y., Sabatini, D. M., Dennis, J. W. & Pawson, T. The adaptor protein p66Shc inhibits mTOR-dependent anabolic metabolism. Sci. Signal. 7, ra17 (2014).
3. Lone, A., Harris, R. A., Singh, O., Betts, D. H. & Cumming, R. C. p66Shc activation promotes increased oxidative phosphorylation and renders CNS cells more vulnerable to amyloid beta toxicity. Sci. Rep. 8, 17081 (2018).
4. Depaoli, M. R., Karsten, F., Madreiter-Sokolowski, C. T., Klec, C., Gottschalk, B., Bischof, H., Eroglu, E., Waldeck-Weiermair, M., Simmen, T., Graier, W. F. & Malli, R. Real-Time Imaging of Mitochondrial ATP Dynamics Reveals the Metabolic Setting of Single Cells. Cell Rep. 25, 501-512.e3 (2018).
5. Dai, Y., Kiselak, T., Clark, J., Clore, E., Zheng, K., Cheng, A., Kujoth, G. C., Prolla, T. A., Maratos-Flier, E. & Simon, D. K. Behavioral and metabolic characterization of heterozygous and homozygous POLG mutator mice. Mitochondrion 13, 282-291 (2013).
6. Alkahest Announces Positive Top-line Data from Phase 2 Study in Mild to Moderate Alzheimer's Disease—Alkahest. Alkahest (2019). at <https://www.alkahest.com/alkahest-announces-positive-top-line-data-from-phase-2-study-in-mild-to-moderate-alzheimers-disease/>
7. Wallace, D. C. A mitochondrial paradigm of metabolic and degenerative diseases, aging, and cancer: a dawn for evolutionary medicine. Annu. Rev. Genet. 39, 359-407 (2005).
8. Shigenaga, M. K., Hagen, T. M. & Ames, B. N. Oxidative damage and mitochondrial decay in aging. Proc. Natl. Acad. Sci. U.S.A 91, 10771-10778 (1994).
9. Balaban, R. S., Nemoto, S. & Finkel, T. Mitochondria, oxidants, and aging. Cell 120, 483-495 (2005).
10. Lin, M. T. & Beal, M. F. Mitochondrial dysfunction and oxidative stress in neurodegenerative diseases. Nature 443, 787-795 (2006).
11. Guarente, L. Mitochondria—a nexus for aging, calorie restriction, and sirtuins? Cell 132, 171-176 (2008).
12. Wallace, D. C. Mitochondrial diseases in man and mouse. Science 283, 1482-1488 (1999).
13. Schapira, A. H. V. Mitochondrial diseases. Lancet 379, 1825-1834 (2012).
14. Pfeffer, G., Majamaa, K., Turnbull, D. M., Thorburn, D. & Chinnery, P. F. Treatment for mitochondrial disorders. Cochrane Database Syst. Rev. Cd004426 (2012). doi:10.1002/14651858.CD004426.pub3
15. Lightowlers, R. N., Taylor, R. W. & Turnbull, D. M. Mutations causing mitochondrial disease: What is new and what challenges remain? Science 349, 1494-1499 (2015).
16. Kujoth, G. C., Hiona, A., Pugh, T. D., Someya, S., Panzer, K., Wohlgemuth, S. E., Hofer, T., Seo, A. Y., Sullivan, R., Jobling, W. A., Morrow, J. D., Van Remmen, H., Sedivy, J. M., Yamasoba, T., Tanokura, M., Weindruch, R., Leeuwenburgh, C. & Prolla, T. A. Mitochondrial DNA mutations, oxidative stress, and apoptosis in mammalian aging. Science 309, 481-484 (2005).
17. Chan, S. S. L. & Copeland, W. C. DNA polymerase gamma and mitochondrial disease: understanding the consequence of POLG mutations. Biochim. Biophys. Acta 1787, 312-319 (2009).
18. Stumpf, J. D., Saneto, R. P. & Copeland, W. C. Clinical and molecular features of POLG-related mitochondrial disease. Cold Spring Harb. Perspect. Biol. 5, a011395 (2013).
19. Stumpf, J. D. & Copeland, W. C. Mitochondrial DNA replication and disease: insights from DNA polymerase gamma mutations. Cell. Mol. Life Sci. 68, 219-233 (2011).
20. Safdar, A., Bourgeois, J. M., Ogborn, D. I., Little, J. P., Hettinga, B. P., Akhtar, M., Thompson, J. E., Melov, S., Mocellin, N. J., Kujoth, G. C., Prolla, T. A. & Tarnopolsky, M. A. Endurance exercise rescues progeroid aging and induces systemic mitochondrial rejuvenation in mtDNA mutator mice. Proc. Natl. Acad. Sci. U.S.A 108, 4135-4140 (2011).
21. Clark-Matott, J., Saleem, A., Dai, Y., Shurubor, Y., Ma, X., Safdar, A., Beal, M. F., Tarnopolsky, M. & Simon, D. K. Metabolomic analysis of exercise effects in the POLG mitochondrial DNA mutator mouse brain. Neurobiol. Aging 36, 2972-2983 (2015).
22. Safdar, A., Khrapko, K., Flynn, J. M., Saleem, A., De Lisio, M., Johnston, A. P., Kratysberg, Y., Samjoo, I. A., Kitaoka, Y., Ogborn, D. I., Little, J. P., Raha, S., Parise, G., Akhtar, M., Hettinga, B. P., Rowe, G. C., Arany, Z., Prolla, T. A. & Tarnopolsky, M. A. Exercise-induced mitochondrial p53 repairs mtDNA mutations in mutator mice. Skelet. Muscle 6, 7 (2016).
23. The State of Aging and Health in America 2013. (Centers for Disease Control and Prevention, 2013).
24. Kim, S. K. Common aging pathways in worms, flies, mice and humans. J. Exp. Biol. 210, 1607-1612 (2007).
25. Vijg, J. & Campisi, J. Puzzles, promises and a cure for ageing. Nature 454, 1065-1071 (2008).
26. Guarente, L. Sirtuins and ageing—new findings. EMBO Rep. 14, 750 (2013).
27. Lopez-Otin, C., Blasco, M. A., Partridge, L., Serrano, M. & Kroemer, G. The hallmarks of aging. Cell 153, 1194-1217 (2013).
28. Ulgherait, M., Rana, A., Rera, M., Graniel, J. & Walker, D. W. AMPK modulates tissue and organismal aging in a non-cell-autonomous manner. Cell Rep. 8, 1767-1780 (2014).
29. Finkel, T. & Holbrook, N. J. Oxidants, oxidative stress and the biology of ageing. Nature 408, 239-247 (2000).
30. Scheibye-Knudsen, M., Mitchell, S. J., Fang, E. F., Iyama, T., Ward, T., Wang, J., Dunn, C. A., Singh, N., Veith, S., Hasan-Olive, M. M., Mangerich, A., Wilson, M. A., Mattson, M. P., Bergersen, L. H., Cogger, V. C., Warren, A., Le Couteur, D. G., Moaddel, R., Wilson, D. M., 3rd, Croteau, D. L., de Cabo, R. & Bohr, V. A. A high-fat diet and NAD(+) activate Sirt1 to rescue premature aging in cockayne syndrome. Cell Metab. 20, 840-855 (2014).
31. Houtkooper, R. H., Mouchiroud, L., Ryu, D., Moullan, N., Katsyuba, E., Knott, G., Williams, R. W. & Auwerx, J. Mitonuclear protein imbalance as a conserved longevity mechanism. Nature 497, 451-457 (2013).
32. Sahin, E. & DePinho, R. A. Axis of ageing: telomeres, p53 and mitochondria. Nat. Rev. Mol. Cell Biol. 13, 397-404 (2012).
33. Scheffler, I. E. Mitochondria. 2nd, (J. Wiley and Sons, Inc., 2008).
34. Wang, Y., Michikawa, Y., Mallidis, C., Bai, Y., Woodhouse, L., Yarasheski, K. E., Miller, C. A., Askanas, V., Engel, W. K., Bhasin, S. & Attardi, G. Muscle-specific mutations accumulate with aging in critical human mtDNA control sites for replication. Proc. Natl. Acad. Sci. U.S.A 98, 4022-4027 (2001).
35. Melov, S., Shoffner, J. M., Kaufman, A. & Wallace, D. C. Marked increase in the number and variety of mitochondrial DNA rearrangements in aging human skeletal muscle. Nucleic Acids Res. 23, 4122-4126 (1995).
36. Corral-Debrinski, M., Horton, T., Lott, M. T., Shoffner, J. M., Beal, M. F. & Wallace, D. C. Mitochondrial DNA deletions in human brain: regional variability and increase with advanced age. Nat. Genet. 2, 324-329 (1992).
37. Guney, O., Ak, H., Atay, S., Ozkaya, A. B. & Aydin, H. H. Mitochondrial DNA polymorphisms associated with longevity in the Turkish population. Mitochondrion 17, 7-13 (2014).
38. Tonna, E. A. & Pillsbury, N. Changes in the osteoblastic and mitochondrial population of aging periosteum. Nature 183, 337-338 (1959).
39. Harman, D. The biologic clock: the mitochondria? J. Am. Geriatr. Soc. 20, 145-147 (1972).
40. Foury, F. & Vanderstraeten, S. Yeast mitochondrial DNA mutators with deficient proofreading exonucleolytic activity. EMBO J. 11, 2717-2726 (1992).
41. Vanderstraeten, S., Van den Brule, S., Hu, J. & Foury, F. The role of 3′-5′ exonucleolytic proofreading and mismatch repair in yeast mitochondrial DNA error avoidance. J. Biol. Chem. 273, 23690-23697 (1998).
42. Trifunovic, A., Wredenberg, A., Falkenberg, M., Spelbrink, J. N., Rovio, A. T., Bruder, C. E., Bohlooly, Y. M., Gidlof, S., Oldfors, A., Wibom, R., Tornell, J., Jacobs, H. T. & Larsson, N. G. Premature ageing in mice expressing defective mitochondrial DNA polymerase. Nature 429, 417-423 (2004).
43. Ahlqvist, K. J., Hamalainen, R. H., Yatsuga, S., Uutela, M., Terzioglu, M., Gotz, A., Forsstrom, S., Salven, P., Angers-Loustau, A., Kopra, O. H., Tyynismaa, H., Larsson, N. G., Wartiovaara, K., Prolla, T., Trifunovic, A. & Suomalainen, A. Somatic progenitor cell vulnerability to mitochondrial DNA mutagenesis underlies progeroid phenotypes in Polg mutator mice. Cell Metab. 15, 100-109 (2012).
44. Conboy, I. M., Conboy, M. J., Wagers, A. J., Girma, E. R., Weissman, I. L. & Rando, T. A. Rejuvenation of aged progenitor cells by exposure to a young systemic environment. Nature 433, 760-764 (2005).
45. Villeda, S. A., Luo, J., Mosher, K. I., Zou, B., Britschgi, M., Bieri, G., Stan, T. M., Fainberg, N., Ding, Z., Eggel, A., Lucin, K. M., Czirr, E., Park, J. S., Couillard-Despres, S., Aigner, L., Li, G., Peskind, E. R., Kaye, J. A., Quinn, J. F., Galasko, D. R., Xie, X. S., Rando, T. A. & Wyss-Coray, T. The ageing systemic milieu negatively regulates neurogenesis and cognitive function. Nature 477, 90-94 (2011).
46. Ruckh, J. M., Zhao, J. W., Shadrach, J. L., van Wijngaarden, P., Rao, T. N., Wagers, A. J. & Franklin, R. J. Rejuvenation of regeneration in the aging central nervous system. Cell Stem Cell 10, 96-103 (2012).
47. Loffredo, F. S., Steinhauser, M. L., Jay, S. M., Gannon, J., Pancoast, J. R., Yalamanchi, P., Sinha, M., Dall'Osso, C., Khong, D., Shadrach, J. L., Miller, C. M., Singer, B. S., Stewart, A., Psychogios, N., Gerszten, R. E., Hartigan, A. J., Kim, M.-J., Serwold, T., Wagers, A. J. & Lee, R. T. Growth differentiation factor 11 is a circulating factor that reverses age-related cardiac hypertrophy. Cell 153, 828-839 (2013).
48. Salpeter, S. J., Khalaileh, A., Weinberg-Corem, N., Ziv, O., Glaser, B. & Dor, Y. Systemic regulation of the age-related decline of pancreatic beta-cell replication. Diabetes 62, 2843-2848 (2013).
49. Sinha, M., Jang, Y. C., Oh, J., Khong, D., Wu, E. Y., Manohar, R., Miller, C., Regalado, S. G., Loffredo, F. S., Pancoast, J. R., Hirshman, M. F., Lebowitz, J., Shadrach, J. L., Cerletti, M., Kim, M.-J., Serwold, T., Goodyear, L. J., Rosner, B., Lee, R. T. & Wagers, A. J. Restoring systemic GDF11 levels reverses age-related dysfunction in mouse skeletal muscle. Science 344, 649-652 (2014).
50. Katsimpardi, L., Litterman, N. K., Schein, P. A., Miller, C. M., Loffredo, F. S., Wojtkiewicz, G. R., Chen, J. W., Lee, R. T., Wagers, A. J. & Rubin, L. L. Vascular and neurogenic rejuvenation of the aging mouse brain by young systemic factors. Science 344, 630-634 (2014).
51. Villeda, S. A., Plambeck, K. E., Middeldorp, J., Castellano, J. M., Mosher, K. I., Luo, J., Smith, L. K., Bieri, G., Lin, K., Berdnik, D., Wabl, R., Udeochu, J., Wheatley, E. G., Zou, B., Simmons, D. A., Xie, X. S., Longo, F. M. & Wyss-Coray, T. Young blood reverses age-related impairments in cognitive function and synaptic plasticity in mice. Nat. Med. 20, 659-663 (2014).
52. Tauchi, H. & Sato, T. Changes in hepatic cell mitochondria during parabiosis between old and young rats. Mech. Ageing Dev. 12, 7-14 (1980).
53. Brack, A. S., Conboy, M. J., Roy, S., Lee, M., Kuo, C. J., Keller, C. & Rando, T. A. Increased Wnt signaling during aging alters muscle stem cell fate and increases fibrosis. Science 317, 807-810 (2007).
54. King, A., Selak, M. A. & Gottlieb, E. Succinate dehydrogenase and fumarate hydratase: linking mitochondrial dysfunction and cancer. Oncogene 25, 4675-4682 (2006).
55. Fulda, S., Galluzzi, L. & Kroemer, G. Targeting mitochondria for cancer therapy. Nat. Rev. Drug Discov. 9, 447-464 (2010).
56. Lesnefsky, E. J., Moghaddas, S., Tandler, B., Kerner, J. & Hoppel, C. L. Mitochondrial dysfunction in cardiac disease: ischemia—reperfusion, aging, and heart failure. J. Mol. Cell. Cardiol. 33, 1065-1089 (2001).
57. Chen, H. & Chan, D. C. Mitochondrial dynamics—fusion, fission, movement, and mitophagy—in neurodegenerative diseases. Hum. Mol. Genet. 18, R169-76 (2009).
58. Valente, E. M., Abou-Sleiman, P. M., Caputo, V., Muqit, M. M. K., Harvey, K., Gispert, S., Ali, Z., Del Turco, D., Bentivoglio, A. R., Healy, D. G., Albanese, A., Nussbaum, R., Gonzalez-Maldonado, R., Deller, T., Salvi, S., Cortelli, P., Gilks, W. P., Latchman, D. S., Harvey, R. J., Dallapiccola, B., Auburger, G. & Wood, N. W. Hereditary early-onset Parkinson's disease caused by mutations in PINK1. Science 304, 1158-1160 (2004).
59. Langston, J. W. & Ballard, P. A., Jr. Parkinson's disease in a chemist working with 1-methyl-4-phenyl-1,2,5,6-tetrahydropyridine. N. Engl. J. Med. 309, 310 (1983).
60. Betarbet, R., Sherer, T. B., MacKenzie, G., Garcia-Osuna, M., Panov, A. V. & Greenamyre, J. T. Chronic systemic pesticide exposure reproduces features of Parkinson's disease. Nat. Neurosci. 3, 1301-1306 (2000).
61. Vanhooren, V. & Libert, C. The mouse as a model organism in aging research: usefulness, pitfalls and possibilities. Ageing Res. Rev. 12, 8-21 (2013).
62. So, B., Kim, H.-J., Kim, J. & Song, W. Exercise-induced myokines in health and metabolic diseases. Integr Med Res 3, 172-179 (2014).
63. Ostrowski, K., Hermann, C., Bangash, A., Schjerling, P., Nielsen, J. N. & Pedersen, B. K. A trauma-like elevation of plasma cytokines in humans in response to treadmill running. J. Physiol. 513 (Pt 3), 889-894 (1998).
64. Ohba, H., Takada, H., Musha, H., Nagashima, J., Mori, N., Awaya, T., Omiya, K. & Murayama, M. Effects of prolonged strenuous exercise on plasma levels of atrial natriuretic peptide and brain natriuretic peptide in healthy men. Am. Heart J. 141, 751-758 (2001).
65. Ostrowski, K., Schjerling, P. & Pedersen, B. K. Physical activity and plasma interleukin-6 in humans—effect of intensity of exercise. Eur. J. Appl. Physiol. 83, 512-515 (2000).
66. Catoire, M., Mensink, M., Kalkhoven, E., Schrauwen, P. & Kersten, S. Identification of human exercise-induced myokines using secretome analysis. Physiol. Genomics 46, 256-267 (2014).
67. Pourteymour, S., Eckardt, K., Holen, T., Langleite, T., Lee, S., Jensen, J., Birkeland, K. I., Drevon, C. A. & Hjorth, M. Global mRNA sequencing of human skeletal muscle: Search for novel exercise-regulated myokines. Mol Metab 6, 352-365 (2017).
68. Colbert, L. H., Davis, J. M., Essig, D. A., Ghaffar, A. & Mayer, E. P. Tissue expression and plasma concentrations of TNFalpha, IL-1beta, and IL-6 following treadmill exercise in mice. Int. J. Sports Med. 22, 261-267 (2001).
69. Ferraiolo, B. L., Mohler, M. A. & Gloff, C. A. Protein Pharmacokinetics and Metabolism. (Springer Science & Business Media, 1992).
70. Someya, S., Kujoth, G. C., Kim, M.-J., Hacker, T. A., Vermulst, M., Weindruch, R. & Prolla, T. A. Effects of calorie restriction on the lifespan and healthspan of POLG mitochondrial mutator mice. PLoS One 12, e0171159 (2017).
71. Lanza, I. R. & Nair, K. S. Mitochondrial metabolic function assessed in vivo and in vitro. Curr. Opin. Clin. Nutr. Metab. Care 13, 511-517 (2010).
72. Doerrier, C., Garcia-Souza, L. F., Krumschnabel, G., Wohlfarter, Y., Mészáros, A. T. & Gnaiger, E. High-Resolution FluoRespirometry and OXPHOS Protocols for Human Cells, Permeabilized Fibers from Small Biopsies of Muscle, and Isolated Mitochondria. Methods Mol. Biol. 1782, 31-70 (2018).
73. Gonzalez-Freire, M., Scalzo, P., D'Agostino, J., Moore, Z. A., Diaz-Ruiz, A., Fabbri, E., Zane, A., Chen, B., Becker, K. G., Lehrmann, E., Zukley, L., Chia, C. W., Tanaka, T., Coen, P. M., Bernier, M., de Cabo, R. & Ferrucci, L. Skeletal muscle ex vivo mitochondrial respiration parallels decline in vivo oxidative capacity, cardiorespiratory fitness, and muscle strength: The Baltimore Longitudinal Study of Aging. Aging Cell 17, (2018).
74. Hiona, A., Sanz, A., Kujoth, G. C., Pamplona, R., Seo, A. Y., Hofer, T., Someya, S., Miyakawa, T., Nakayama, C., Samhan-Arias, A. K., Servais, S., Barger, J. L., Portero-Otín, M., Tanokura, M., Prolla, T. A. & Leeuwenburgh, C. Mitochondrial DNA mutations induce mitochondrial dysfunction, apoptosis and sarcopenia in skeletal muscle of mitochondrial DNA mutator mice. PLoS One 5, e11468 (2010).
75. Joseph, A.-M., Adhihetty, P. J., Wawrzyniak, N. R., Wohlgemuth, S. E., Picca, A., Kujoth, G. C., Prolla, T. A. & Leeuwenburgh, C. Dysregulation of mitochondrial quality control processes contribute to sarcopenia in a mouse model of premature aging. PLoS One 8, e69327 (2013).
76. Dillon, L. M., Hida, A., Garcia, S., Prolla, T. A. & Moraes, C. T. Long-term bezafibrate treatment improves skin and spleen phenotypes of the mtDNA mutator mouse. PLoS One 7, e44335 (2012).
77. Isaacs, A. & Lindenmann, J. Virus interference. I. The interferon. Proc. R. Soc. Lond. B Biol. Sci. 147, 258-267 (1957).
78. Dhariwal, A. P., Antunesrodrigues, J., Reeser, F., Chowers, I. & McCann, S. M. Purification of hypothalamic corticotrophin-releasing factor (CRF) of ovine origin. Proc. Soc. Exp. Biol. Med. 121, 8-12 (1966).
79. Dhariwal, A. P., Krulich, L. & McCann, S. M. Purification of a growth hormone-inhibiting factor (CIF) from sheep hypothalamus. Neuroendocrinology 4, 282-288 (1969).
80. Katz, Y. J. & Goldblatt, H. STUDIES ON EXPERIMENTAL HYPERTENSION: XXI. THE PURIFICATION OF RENIN. J. Exp. Med. 78, 67-74 (1943).
81. Deutsch, H. F., Gosting, L. J. & et al. Biophysical studies of blood plasma proteins; recovery of gamma-globulin from human blood protein mixtures. J. Biol. Chem. 164, 109-118 (1946).
82. Rapport, M. M., Green, A. A. & Page, I. H. Partial purification of the vasoconstrictor in beef serum. J. Biol. Chem. 174, 735-741 (1948).
83. Wu, Z. D., Puigserver, P., Andersson, U., Zhang, C. Y., Adelmant, G., Mootha, V., Troy, A., Cinti, S., Lowell, B., Scarpulla, R. C. & Spiegelman, B. M. Mechanisms controlling mitochondrial biogenesis and respiration through the thermogenic coactivator PGC-1. Cell 98, 115-124 (1999).
84. Handschin, C., Rhee, J., Lin, J., Tarr, P. T. & Spiegelman, B. M. An autoregulatory loop controls peroxisome proliferator-activated receptor gamma coactivator 1alpha expression in muscle. Proc. Natl. Acad. Sci. U.S.A 100, 7111-7116 (2003).
85. Yin, W., Signore, A. P., Iwai, M., Cao, G., Gao, Y. & Chen, J. Rapidly increased neuronal mitochondrial biogenesis after hypoxic-ischemic brain injury. Stroke 39, 3057-3063 (2008).
86. Ripolone, M., Ronchi, D., Violano, R., Vallejo, D., Fagiolari, G., Barca, E., Lucchini, V., Colombo, I., Villa, L., Berardinelli, A., Balottin, U., Morandi, L., Mora, M., Bordoni, A., Fortunato, F., Corti, S., Parisi, D., Toscano, A., Sciacco, M., DiMauro, S., Comi, G. P. & Moggio, M. Impaired Muscle Mitochondrial Biogenesis and Myogenesis in Spinal Muscular Atrophy. JAMA Neurol. 72, 666-675 (2015).
87. Katayama, H., Kogure, T., Mizushima, N., Yoshimori, T. & Miyawaki, A. A sensitive and quantitative technique for detecting autophagic events based on lysosomal delivery. Chem. Biol. 18, 1042-1052 (2011).
88. Sun, N., Malide, D., Liu, J., Rovira, I. I., Combs, C. A. & Finkel, T. A fluorescence-based imaging method to measure in vitro and in vivo mitophagy using mt-Keima. Nat. Protoc. 12, 1576-1587 (2017).
89. Ikeda, Y., Shirakabe, A., Maejima, Y., Zhai, P., Sciarretta, S., Toli, J., Nomura, M., Mihara, K., Egashira, K., Ohishi, M., Abdellatif, M. & Sadoshima, J. Endogenous Drpl Mediates Mitochondrial Autophagy and Protects the Heart Against Energy Stress. Circ. Res. (2014). doi:10.1161/circresaha.116.303356
90. Beeson, C. C., Beeson, G. C. & Schnellmann, R. G. A high-throughput respirometric assay for mitochondrial biogenesis and toxicity. Anal. Biochem. 404, 75-81 (2010).
91. Wu, M., Neilson, A., Swift, A. L., Moran, R., Tamagnine, J., Parslow, D., Armistead, S., Lemire, K., Orrell, J., Teich, J. & Others. Multiparameter metabolic analysis reveals a close link between attenuated mitochondrial bioenergetic function and enhanced glycolysis dependency in human tumor cells. American Journal of Physiology-Cell Physiology 292, C125-C136 (2007).
92. Pendergrass, W., Wolf, N. & Poot, M. Efficacy of MitoTracker Green and CMXrosamine to measure changes in mitochondrial membrane potentials in living cells and tissues. Cytometry A 61, 162-169 (2004).
93. Reitzer, L. J., Wice, B. M. & Kennell, D. Evidence that glutamine, not sugar, is the major energy source for cultured HeLa cells. J. Biol. Chem. 254, 2669-2676 (1979).
94. Rossignol, R., Gilkerson, R., Aggeler, R., Yamagata, K., Remington, S. J. & Capaldi, R. A. Energy substrate modulates mitochondrial structure and oxidative capacity in cancer cells. Cancer Res. 64, 985-993 (2004).
95. Marroquin, L. D., Hynes, J., Dykens, J. A., Jamieson, J. D. & Will, Y. Circumventing the Crabtree effect: replacing media glucose with galactose increases susceptibility of HepG2 cells to mitochondrial toxicants. Toxicol. Sci. 97, 539-547 (2007).
96. Gohil, V. M., Sheth, S. A., Nilsson, R., Wojtovich, A. P., Lee, J. H., Perocchi, F., Chen, W., Clish, C. B., Ayata, C., Brookes, P. S. & Mootha, V. K. Nutrient-sensitized screening for drugs that shift energy metabolism from mitochondrial respiration to glycolysis. Nat. Biotechnol. 28, 249-255 (2010).
97. Robinson, B. H., Petrova-Benedict, R., Buncic, J. R. & Wallace, D. C. Nonviability of cells with oxidative defects in galactose medium: a screening test for affected patient fibroblasts. Biochem. Med. Metab. Biol. 48, 122-126 (1992).
98. Wang, R., Novick, S. J., Mangum, J. B., Queen, K., Ferrick, D. A., Rogers, G. W. & Stimmel, J. B. The acute extracellular flux (XF) assay to assess compound effects on mitochondrial function. J. Biomol. Screen. 20, 422-429 (2015).
99. Andreux, P. A., Houtkooper, R. H. & Auwerx, J. Pharmacological approaches to restore mitochondrial function. Nat. Rev. Drug Discov. 12, 465-483 (2013).
100. Vermulst, M., Bielas, J. H., Kujoth, G. C., Ladiges, W. C., Rabinovitch, P. S., Prolla, T. A. & Loeb, L. A. Mitochondrial point mutations do not limit the natural lifespan of mice. Nat. Genet. 39, 540-543 (2007).
101. Vermulst, M., Wanagat, J., Kujoth, G. C., Bielas, J. H., Rabinovitch, P. S., Prolla, T. A. & Loeb, L. A. DNA deletions and clonal mutations drive premature aging in mitochondrial mutator mice. Nat. Genet. 40, 392-394 (2008).
102. Taylor, S. D., Ericson, N. G., Burton, J. N., Prolla, T. A., Silber, J. R., Shendure, J. & Bielas, J. H. Targeted enrichment and high-resolution digital profiling of mitochondrial DNA deletions in human brain. Aging Cell 13, 29-38 (2014).
103. Pinheiro, L. B., Coleman, V. A., Hindson, C. M., Herrmann, J., Hindson, B. J., Bhat, S. & Emslie, K. R. Evaluation of a droplet digital polymerase chain reaction format for DNA copy number quantification. Anal. Chem. 84, 1003-1011 (2012).
104. Belmonte, F. R., Martin, J. L., Frescura, K., Damas, J., Pereira, F., Tarnopolsky, M. A. & Kaufman, B. A. Digital PCR methods improve detection sensitivity and measurement precision of low abundance mtDNA deletions. Sci. Rep. 6, 25186 (2016).
105. Wanagat, J., Ahmadieh, N., Bielas, J. H., Ericson, N. G. & Van Remmen, H. Skeletal muscle mitochondrial DNA deletions are not increased in CuZn-superoxide dismutase deficient mice. Exp. Gerontol. 61, 15-19 (2015).
106. Williams, J. A., Zhao, K., Jin, S. & Ding, W.-X. New methods for monitoring mitochondrial biogenesis and mitophagy in vitro and in vivo. Exp. Biol. Med. 242, 781-787 (2017).
107. Bingol, B., Tea, J. S., Phu, L., Reichelt, M., Bakalarski, C. E., Song, Q., Foreman, O., Kirkpatrick, D. S. & Sheng, M. The mitochondrial deubiquitinase USP30 opposes parkin-mediated mitophagy. Nature 510, 370-375 (2014).
108. Kageyama, Y., Hoshijima, M., Seo, K., Bedja, D., Sysa-Shah, P., Andrabi, S. A., Chen, W., Höke, A., Dawson, V. L., Dawson, T. M., Gabrielson, K., Kass, D. A., lijima, M. & Sesaki, H. Parkin-independent mitophagy requires Drpl and maintains the integrity of mammalian heart and brain. EMBO J. 33, 2798-2813 (2014).
109. Mizumura, K., Cloonan, S. M., Nakahira, K., Bhashyam, A. R., Cervo, M., Kitada, T., Glass, K., Owen, C. A., Mahmood, A., Washko, G. R., Hashimoto, S., Ryter, S. W. & Choi, A. M. K. Mitophagy-dependent necroptosis contributes to the pathogenesis of COPD. J. Clin. Invest. 124, 3987-4003 (2014).
110. Vande Voorde, J., Ackermann, T., Pfetzer, N., Sumpton, D., Mackay, G., Kalna, G., Nixon, C., Blyth, K., Gottlieb, E. & Tardito, S. Improving the metabolic fidelity of cancer models with a physiological cell culture medium. Science Advances 5, eaau7314 (2019).
111. Echave, P., Machado-da-Silva, G., Arkell, R. S., Duchen, M. R., Jacobson, J., Mitter, R. & Lloyd, A. C. Extracellular growth factors and mitogens cooperate to drive mitochondrial biogenesis. J. Cell Sci. 122, 4516-4525 (2009).
112. Scarpulla, R. C. Metabolic control of mitochondrial biogenesis through the PGC-1 family regulatory network. Biochim. Biophys. Acta 1813, 1269-1278 (2011).
113. Quiros, P. M., Mottis, A. & Auwerx, J. Mitonuclear communication in homeostasis and stress. Nat. Rev. Mol. Cell Biol. 17, 213-226 (2016).
114. Shirihai, O. S., Song, M. & Dorn, G. W., 2nd. How mitochondrial dynamism orchestrates mitophagy. Circ. Res. 116, 1835-1849 (2015).
115. Nguyen, T. N., Padman, B. S. & Lazarou, M. Deciphering the Molecular Signals of PINK1/Parkin Mitophagy. Trends Cell Biol. (2016). doi:10.1016/j.tcb.2016.05.008
116. Yamaguchi, O., Murakawa, T., Nishida, K. & Otsu, K. Receptor-mediated mitophagy. J. Mol. Cell. Cardiol. 95, 50-56 (2016).
117. Twig, G., Elorza, A., Molina, A. J., Mohamed, H., Wikstrom, J. D., Walzer, G., Stiles, L., Haigh, S. E., Katz, S., Las, G., Alroy, J., Wu, M., Py, B. F., Yuan, J., Deeney, J. T., Corkey, B. E. & Shirihai, O. S. Fission and selective fusion govern mitochondrial segregation and elimination by autophagy. EMBO J. 27, 433-446 (2008).
118. Meeusen, S., McCaffery, J. M. & Nunnari, J. Mitochondrial fusion intermediates revealed in vitro. Science 305, 1747-1752 (2004).

What is claimed is:

1. A method of treating or preventing a mitochondrial or age-related disease or disorder in a subject in need thereof, comprising administering an effective amount of an exercised blood product to the subject.

2. The method of claim 1, wherein the exercised blood product comprises factors that signal mitochondria to cause regeneration of healthy cells, stem cells and/or progenitor cells.

3. The method of claim 2, wherein the factors comprise small molecules or large molecules.

4. The method of claim 3, wherein the small molecules comprise microRNA, metabolites, or steroids.

5. The method of claim 3, wherein the large molecules comprises proteins.

6. The method of claim 5, wherein the proteins have a molecular weight of no more than 30 kDa or no more than 20 kDa.

7. The method of claim 5 or claim 6, wherein the proteins comprise cytokines, hormones and/or growth factors.

8. The method of claim 7, wherein the proteins comprise cytokines.

9. The method of claim 8, wherein the cytokines comprise adipokines, chemokines, colony-stimulating factors, interferons, interleukins, monokines, myokines and/or lymphokines.

10. The method of any one of claims 1-9, wherein the exercised blood product has been obtained from a blood sample from a donor animal that has been subjected to exercise.

11. The method of claim 10, wherein the exercise comprises endurance exercise, rigorous exercise, and/or resistance exercise.

12. The method of claim 10 or claim 11, wherein the donor animal is young.

13. The method of any one of claims 10-12, wherein the donor animal is a mammal.

14. The method of claim 13, wherein the mammal is a human.

15. The method of any one of claims 1-14, further comprising monitoring the subject for improved mitochondrial fitness or function.

16. The method of any one of claims 1-15, wherein the subject is a mammal.

17. The method of claim 16, wherein the mammal is a human.

18. The method of any one of claims 1-17, wherein the exercised blood product is administered at least once per week, or at least twice per week, or at least three times per week.

19. The method of any one of claims 1-19, wherein the exercised blood product is administered for at least 2, 3, 4, 5, 6, 7, 8, 9, or 10 weeks.

20. The method of claim 19, wherein the exercised blood product is administered three times per week for at least 4 weeks.

21. The method of claim 19, wherein the exercised blood product is administered three times per week for at least 8 weeks.

22. The method of any one of claims 1-21, wherein the administration is by injection.

23. The method of claim 22, wherein the administration is by intravenous injection.

24. The method of any one of claims 1-23, wherein the exercised blood product comprises plasma components.

25. The method of any one of claims 1-24, wherein the exercised blood product is plasma.

26. The method of any one of claims 1-24, wherein the exercised blood product is a plasma fraction.

27. The method of any one of claims 1-24, wherein the mitochondrial or age-related disease or disorder involves mitochondrial dysfunction.

28. The method of any one of claim 27, wherein the mitochondrial or age-related disease or disorder involving mitochondrial dysfunction comprises a mitochondrial disease, muscle disorder, cardiovascular disease, autoimmune disease, NF-kappaB mediated disease, respiratory disease, neurodegeneration or neuroinflammation, and/or demyelinating neurological disorder.

29. The method of claim 28, wherein the mitochondrial disease is Leber's hereditary optic neuropathy, MELAS (Mitochondrial Encephalomyopathy; Lactic Acidosis; Stroke), MERRF (Myoclonic Epilepsy; Ragged Red Fibers), progressive external opthalmoplegia, Leigh's syndrome, MNGIE (Myopathy and external ophthalmoplegia; Neuropathy; Gastro-Intestinal; Encephalopathy), Kearns-Sayre Syndrome, NARP (Neuropathy, ataxia, and retinitis pigmentosa), Hereditary Spastic Paraparesis, Mitochondrial myopathy, Friedreich ataxia, retinopathia pigmentosa, and/or a form of mitochondrial encephalomyopathy.

30. The method of claim 28, wherein the muscle disorder is sarcopenia, frailty, nemaline myopathy, Spinocerebellar ataxia, Spinal muscular atrophy, or deconditioning from inactivity, hospitalization, and/or any surgical procedure.

31. A pharmaceutical composition for treating, retarding the progression of, delaying the onset of, prophylaxis of, amelioration of, or reducing the symptoms of mitochondrial dysfunction in a subject comprising an effective amount of exercise-derived blood products.

32. The pharmaceutical composition of claim 31, wherein the mitochondrial dysfunction is associated with a mitochondrial disease, muscle disorder, cardiovascular disease, autoimmune disease, NF-kappaB mediated disease, respiratory disease, neurodegeneration or neuroinflammation, and/or demyelinating neurological disorder.

33. The pharmaceutical composition of claim 32, wherein the mitochondrial disease is Leber's hereditary optic neuropathy, MELAS (Mitochondrial Encephalomyopathy; Lactic Acidosis; Stroke), MERRF (Myoclonic Epilepsy; Ragged Red Fibers), progressive external opthalmoplegia, Leigh's syndrome, MNGIE (Myopathy and external ophthalmoplegia; Neuropathy; Gastro-Intestinal; Encephalopathy), Kearns-Sayre Syndrome, NARP (Neuropathy, ataxia, and retinitis pigmentosa), Hereditary Spastic Paraparesis, Mitochondrial myopathy, Friedreich ataxia, retinopathia pigmentosa, and/or a form of mitochondrial encephalomyopathy.

34. The pharmaceutical composition of claim 32, wherein the muscle disorder is sarcopenia, frailty, nemaline myopathy, Spinocerebellar ataxia, Spinal muscular atrophy, or deconditioning from inactivity, hospitalization, and/or any surgical procedure.
